SEQUENCE LISTING (2 sequences)

SEQ ID NO: 1 - DNA, 20 bp, Artificial Sequence
  Description of Artificial Sequence Synthetic primer
tagggcaaaccatctggaag

SEQ ID NO: 2 - DNA, 20 bp, Artificial Sequence
  Description of Artificial Sequence Synthetic primer
acttggagctacaggcctca

(19) United States
(12) Patent Application Publication
(10) Pub. No.: US 2009/0252785 A1
(43) Pub. Date: Oct. 8, 2009

(54) ENDOPLASMIC RETICULUM TARGETING LIPOSOMES

(75) Inventors: Stephanie Pollock, Oxford (GB); Raymond Allen Dwek, Oxford (GB); Nicole Zitzmann, Oxford (GB)

Correspondence Address: FOLEY AND LARDNER LLP;SUITE 500, 3000 K STREET NW, WASHINGTON, DC, 20007, US

(73) Assignee: University of Oxford

(21) Appl. No.: 12/410,750

(22) Filed: Mar. 25, 2009

Related U.S. Application Data
(60) Provisional application No. 61/039,638, filed on Mar. 26, 2008.

(51) Int. Cl. A61K 9/127; A61K 31/70; C12Q 1/70

(52) U.S. Cl. 424/450; 514/23; 435/5; 435/375

(57) ABSTRACT

Provided are compositions that include lipid particles, such as liposomes, that can fuse with the ER membrane of a cell. The lipid particles can also deliver a cargo, such as a therapeutic or an imaging agent, encapsulated inside the particles inside the ER lumen of the cell. The compositions can be useful for treating and/or preventing diseases or conditions caused by or associated with a virus, such as viral infections, including HIV and HCV infections.

### RELATED APPLICATION

The present application claims priority to U.S. provisional patent application No. 61/039,638 filed Mar. 26, 2008, which is incorporated herein by reference in its entirety.

### FIELD

The present application relates generally to methods and compositions for delivery active agents, such as therapeutic agents and/or imaging agents and, more specifically, to methods and compositions for delivery active agents utilizing lipid particles, such as liposomes.

### SUMMARY

One embodiment provides a method of drug delivery, comprising administering to a host in need thereof a composition comprising a lipid particle comprising at least one PS lipid.

Another embodiment provides a method of treating or preventing an HIV infection comprising administering to a host in need thereof a composition comprising a lipid particle comprising at least one of PS lipids or PI lipids, wherein said lipid particle does not contain CHEMS lipids.

Yet another embodiment provides a method of drug delivery comprising administering to a subject in need thereof a composition comprising a lipid particle comprising at least one polyunsaturated lipid.

And yet another embodiment provides a composition comprising a lipid particle that comprises PS lipids.

And yet another embodiment provides a composition comprising a lipid particle that comprises at least one polyunsaturated lipid.

And yet still another embodiment is a method of labeling a virus comprising contacting a cell infected with the virus with a lipid particle comprising a) at least one of PI or PS lipids and b) at least one labeled lipid comprising at least one label, wherein said contacting results in labeling said virus with said label.

### DRAWINGS

FIGS. 1 (A)-(G) depict chemical structures of the following lipids: (A) DOPE; (B) DOPC; (C)CHEMS; (D) PI; (E) PS; (F) Rho-PE and (G) b-PE.

FIGS. 2 (A)-(H) present confocal microscope images studying a co-localization of the following liposomes with the ER membrane protein EDEM in Huh7.5 cells: (A) PE:CH (molar ratio 3:2) liposomes; (B) PE:PC (3:2) liposomes; (C) PE:CH:PI (3:1:1) liposomes; (D) PE:PC:PI (2:2:1) liposomes; (E) PE:CH:PS (3:1:1) liposomes; (F) PE:PC:PS (2:2:1) liposomes; (G) PE:CH:PI:PS (3:1:0.5:0.5) liposomes; (H) PE:PC:PI:PS (1.5:1.5:1:1) liposomes.

FIG. 3 presents calculated co-localization of liposome-delivered rh-DOPE with the EDEM antibody.

FIG. 4 displays the percentage of tagged viral particles captured by streptavidin in relation to the total amount of secreted virions within the same sample (100%) as a function of a molar percentage of b-PE in liposomes.

FIG. 5 shows confocal microscope images of Rh-PE-tagged JC-1 HCVcc (red, bottom-left panels) that was incubated with naïve Huh7.5 cells for 1 h (MOI=0.1), following which cells were washed and incubated for a further 0, 6, or 24 h in fresh media. After each incubation time, cells were fixed and stained with an anti-HCV core antibody (green, top-right panel) and DAPI (blue, top-left panel) prior to mounting onto microscope slides and confocal microscopy imaging. Merged images are shown in the bottom-right panels. Representative images from each incubation period are shown. The resolution bar for each image is 10 μm.

FIGS. 6 (A)-(C) present fluorescent microscopy images studying incorporation into cellular membranes of PE:CH liposomes with molar ratio 3:2 (A); PE:CH:PI (3:1:1) liposomes (B) and PE:CH:PS (3:1:1) liposomes (C).

FIG. 7 shows is a plot demonstrating increased cellular uptake and lipid retention of ER-targeting liposomes inside Huh7.5 cells. Data represent the mean and standard deviation (SD) of triplicate samples from three independent experiments. The graph represents two sets of data, cell growth (dotted lines) and rh-PE-liposome uptake (solid lines) for both ER liposomes (black lines) and pH-sensitive liposomes (red lines). The Y-axis represents the maximum value for those two sets of data normalized to 100%. The maximum value of 100% cell growth is 2.4×10 e6 cells/ml (72 h reading with ER liposomes), and the maximum value for rh-PE fluorescence is 1.5×10 e-3 arbitrary units (AU)/cell (96 h reading with ER liposomes). FIG. 8(A) is a plot representing the percentage of calcein released from liposomes in relation to the maximum fluorescence which is determined by the addition of Triton X-100 to disrupt the liposome membranes at the end of the incubation period as a function of time for PE:CH and PE:PC:PI:PS liposomes.

FIG. 8(B) presents results of experiment for Rh-labeled liposomes (50 μm lipid concentration) that were incubated with Huh 7.5 cells for 24 h in the presence of either 10% bovine serum (FBS); 10% human serum or in serum-free media. Following the incubation time, cells were harvested, counted, and fluorescence was measured at λex=550 nm, λem=590 nm. Results are presented as the measured average fluorescence per cell for each sample. All data represent the mean and SD of triplicate samples from three independent experiments.

FIG. 9 shows viability of Huh7.5 cells following a 5 day incubation with different liposome formulations encapsulating 1×PBS. Final lipid concentrations in the medium ranged from 0 to 500 μM. Results represent the mean values of triplicate samples from three independent experiments.

FIG. 10 demonstrates viability of PBMCs following a 5 day incubation with different liposome formulations encapsulating 1×PBS. Final lipid concentrations in the medium ranged from 0 to 500 μM. Results represent the mean values of triplicate samples from three independent experiments.

FIG. 11 presents secretion of HIV from infected PBMCs during treatment with liposomes for 5 days.

FIG. 12 presents the infectivity of HIV virions secreted from liposome-treated HIV-infected PBMCs.

FIG. 13 presents results for experiments for self-quenching calcein-loaded, rh-PE-labeled, liposomes (final lipid concentration of 50 μM) that were incubated with Huh7.5 cells in complete DMEM/10% FBS for 45 min. Intracellular dequenching of calcein from liposomes following the incubation was measured at λex=490 nm, λem=520 nm, and the total liposome uptake during the same incubation period was determined by fluorescent measurements at λex=550 nm, λem=590 nm. The assay was conducted both at 37° C. and 4° C., and to correct for liposome binding without endocytosis, all 4° C. values were subtracted from the 37° C. values. The ability of liposomes to deliver encapsulated calcein inside Huh7.5 cells was measured by calculating the ratio of calcein dequenching and rh-PE fluorescence in treated cells following the incubation. Data represent the mean and SD of triplicate samples from three independent experiments.

FIG. 14 presents secretion of HIV from infected PBMCs during a 5 day treatment with 1 mM NB-DNJ: free vs. liposome-mediated delivery.

FIG. 15 shows the infectivity of HIV virions secreted from NB-DNJ-liposome or free NB-DNJ-treated HIV-infected PBMCs.

FIG. 16 demonstrates viability of PBMCs following a 5 day incubation with different liposome formulations encapsulating 1 mM NB-DNJ.

FIG. 17 presents a secretion of HCV from both acutely and chronically-infected, Huh7.5 cells following treatment with liposomes for 5 days.

FIG. 18 demonstrates the infectivity of HCV virions secreted from liposome-treated HCV-infected Huh7.5 cells, which were infected both acutely and chronically.

FIG. 19 shows confocal microscope images of untreated Huh7.5 cells (left panel) and PE:PC:PI:PS liposome-treated Huh7.5 cells, which were probed with BODIPY 493/503 (green) to visualize LDs following 16 hours of incubation.

FIG. 20 shows confocal microscope images of Huh7.5 cells (left panel) treated with PE:PC:PI:PS liposomes for 2 hours and probed with a LD stain (green). PE:PC:PI:PS liposomes were added to the cell culture media to a final lipid cincentration of 50 μM. DAPI (blue) is used as a nuclear stain. Bottom-right panel is the merged image. Yellow colour identifies areas of co-localization within the cell.

FIG. 21A shows confocal microscope images of untreated Huh7.5 cells (left panel) and PE:PC:PI:PS liposome-treated Huh7.5 cells (right panel), which were incubated for 16 h and probed with an anti-HCV core antibody (red) and an LD stain (green). FIG. 21B shows close-ups of merged images (white boxes in FIG. 9A) for both untreated (left) and PE:PC:PI:PS (right) cells. FIG. 21C is a schematic representation of the HCV core protein/LD interaction in the presence (right) and absence (left) of PE:PC:PI:PS liposomes.

FIGS. 22A-22D present chemical structures of exemplary polyunsaturated lipids: 22:6 PE (A); 20:4 PE (B); 22:6 PC(C) and 20:4 PC (D). FIGS. 23A-B show respectively (23A) JC-1 HCVcc secretion from infected Huh7.5 cells (MOI=0.5) during a 4 day incubation in the presence of various ER liposome formulations was quantified from 500 μl of cellular supernatant. Secretion is measured by the quantification of JC-1 HCVcc RNA within the supernatant by quantitative PCR. (23B) Infectivity of secreted JC-1 HCVcc from liposome-treated, JC-1-infected Huh7.5 cells. Infectivity of the secreted HCVcc was determined by infection of naïve Huh7.5 cells for 1 h, followed by a 48 h incubation at which point cells were fixed and stained with an anti-HCV core antibody to quantify the number of infected cells, and DAPI to visualize all cells.

### DETAILED DESCRIPTION

### Definition of Terms

Unless otherwise specified, “a” or “an” means “one or more.”

As used herein, the term “viral infection” can refer to a diseased state, in which a virus invades a healthy cell, uses the cell's reproductive machinery to multiply or replicate and ultimately lyse the cell resulting in cell death, release of viral particles and the infection of other cells by the newly produced progeny viruses. Latent infection by certain viruses is also a possible result of viral infection.

As used herein, the term “treating or preventing viral infection” can mean inhibiting the replication of the particular virus, inhibiting viral transmission, or preventing the virus from establishing itself in its host, and ameliorating or alleviating the symptoms of the disease caused by the viral infection. The treatment can be considered therapeutic if it results in a reduction in viral load, decrease in mortality and/or morbidity.

The term “therapeutic agent” refers to an agent, such as a molecule or a compound, which can assist in treating a physiological condition, such as a viral infection or a disease caused thereby.

The term “liposome” can be defined a particle comprising lipids in a bilayer formation, which is usually a spherical bilayer formation. Liposomes discussed herein may include one or more lipids represented by the following abbreviations:

CHEMS stands for cholesteryl hemisuccinate lipid.

DOPE stands for dioleoylphosphatidylethanolamine lipid.

DOPC stands for dioleoylphosphatidylcholine lipid.

PE stands for phosphatidylethanolamine lipid or its derivative.

PEG-PE stands for PE lipid conjugated with polyethylene glycol (PEG). One example of PEG-PE can be polyethylene glycol-distearoylphosphatidylethanolamine lipid. Molecular weight of PEG component of PEG can vary.

Rh-PE stands for lissamine rhodamine B-phosphatidylethanolamine lipid.

MCC-PE stands for 1,2-Dioleoyl-sn-Glycero-3-Phosphoethanolamine-N-[4-(p-maleimidomethyl)cyclohexane-carboxamide] lipid.

PI stands for phosphatidylinositol lipid.

PS stands for phosphatidylserine lipid.

The term “intracellular delivery” can refer to the delivery of encapsulated material from liposomes into any intracellular compartment.

IC50 or IC90 (inhibitory concentration 50 or 90) can refer to a concentration of a therapeutic agent used to achieve 50% or 90% reduction of viral infection, respectively.

PBMC stands for peripheral blood mononuclear cell.

sCD4 stands for a soluble CD4 molecule. By “soluble CD4” or “sCD4” or D1D2” is meant a CD4 molecule, or a fragment thereof, that is in aqueous solution and that can mimic the activity of native membrane-anchored CD4 by altering the conformation of HIV Env, as is understood by those of ordinary skill in the art. One example of a soluble CD4 is the two-domain soluble CD4 (sCD4 or D1D2) described, e.g., in Salzwedel et al. J. Virol. 74:326 333, 2000.

MAb stands for a monoclonal antibody.

DNJ denotes deoxynojirimycin.

NB-DNJ denotes N-butyl deoxynojirimycin.

NN-DNJ denotes N-nonyl deoxynojirimycin.

BVDV stands for bovine viral diarrhea virus.

HBV stands for hepatitis B virus.

HCV stands for hepatitis C virus.

HIV stands for human immunodeficiency virus.

Ncp stands for non-cytopathic.

Cp stands for cytopathic.

ER stands for endoplasmic reticulum.

CHO stands for Chinese hamster ovary cells

MDBK stands for Madin-Darby bovine kidney cells.

PCR stands for polymerase chain reaction.

FOS stands for free oligosaccharides.

HPLC stands for high performance liquid chromatography.

PHA stands for phytohemagglutinin.

FBS stands for fetal bovine serum.

TCID50 stands for 50% tissue culture infective dose. ELISA stands for Enzyme Linked Immunosorbent Assay.

IgG stands for immunoglobuline.

DAPI stands for 4′,6-Diamidino-2-phenylindole.

PBS stands for phosphate buffered saline.

LD stands for lipid droplet.

NS stands for non-structural.

“MOI” refers to multiplicity of infection.

### Related Applications

The present disclosure incorporates by reference in its entirety US patent application publication no. 2008-0138351.

### Hepatitis C

Approximately 170 million people worldwide, i.e. 3% of the world's population, see e.g. WHO, J. Viral. Hepat. 1999; 6: 35-47, and approximately 4 million people in the United States are infected with Hepatitis C virus (HCV, HepC). About 80% of individuals acutely infected with HCV become chronically infected. Hence, HCV is a major cause of chronic hepatitis. Once chronically infected, the virus is almost never cleared without treatment. In rare cases, HCV infection causes clinically acute disease and even liver failure. Chronic HCV infection can vary dramatically between individuals, where some will have clinically insignificant or minimal liver disease and never develop complications and others will have clinically apparent chronic hepatitis and may go on to develop cirrhosis. About 20% of individuals with HCV who do develop cirrhosis will develop end-stage liver disease and have an increased risk of developing primary liver cancer.

Antiviral drugs such as interferon, alone or in combination with ribavirin, are effective in up to 80% of patients (Di Bisceglie, A. M, and Hoofnagle, J. H. 2002, Hepatology 36, S121-S127), but many patients do not tolerate this form of combination therapy.

### Lipid Droplets

The lipid droplet (LD) can be an organelle that can be used for the storage of neutral lipids. LD can dynamically move through the cytoplasm, interacting with other organelles, including the ER. These interactions are thought to facilitate the transport of lipids and proteins to other organelles. HCV capsid protein (core) can associate with the cellular LDs and actively recruit non-structural (NS) proteins and replication complexes to LD-associated membranes for the production of infectious viral particles. HCV particles have been observed in close proximity to LDs, indicating that some steps of virus assembly can take place around LDs (Miyanari et al, Nature Cell Biology, 9 (2007) pp. 1089-1097).

### Human Immunodeficiency Virus (HIV)

HIV is the causative agent of acquired immune deficiency syndrome (AIDS) and related disorders. There are at least two distinct types of HIV: HIV-1 and HIV-2. Further, a large amount of genetic heterogeneity exists within populations of each of these types. Since the onset of the AIDS epidemic, some 20 million people have died and the estimate is that over 40 million are now living with HIV-1/AIDS, with 14 000 people infected daily worldwide.

Numerous antiviral therapeutic agents and diagnostic capabilities have been developed that, at least for those with access, have greatly improved both the quantity and quality of life. Most of these drugs interfere with viral proteins or processes such as reverse transcription and protease activity. Unfortunately, these treatments do not eliminate infection, the unwanted effects of many therapies are severe, and drug resistant strains of HIV exist for every type of antiviral currently in use.

### N-butyl-1,5-dideoxy-1,5-imino-D-glucitol

NB-DNJ, also known as N-butyl-1,5-dideoxy-1,5-imino-D-glucitol, can inhibit processing by the ER glucosidases I and II, and has been shown to be an effective antiviral by causing the misfolding and/or ER-retention of glycoproteins of HIV and hepatitis viruses such as Hepatitis B virus (HBV), Hepatitis C virus (HCV), Bovine viral diarrhea virus (BVDV) amongst others. Methods of synthesizing NB-DNJ and other N-substituted deoxynojirimycin derivatives are described, for example, in U.S. Pat. Nos. 5,622,972, 4,246,345, 4,266,025, 4,405,714 and 4,806,650. Antiviral effects of NB-DNJ are discussed, for example, in U.S. Pat. Nos. 6,465,487; 6,545,021; 6,689,759; 6,809,083 for hepatitis viruses and U.S. Pat. No. 4,849,430 for HIV virus.

Glucosidase inhibitors, such as NB-DNJ, have been shown to be effective in the treatment of HBV infection in both cell culture and using a woodchuck animal model, see e.g. T. Block, X. Lu, A. S. Mehta, B. S. Blumberg, B. Tennant, M. Ebling, B. Korba, D. M. Lansky, G. S. Jacob & R. A. Dwek, Nat. Med. 1998 May; 4(5):610-4. NB-DNJ suppresses secretion of HBV particles and causes intracellular retention of HBV DNA.

NB-DNJ has been shown to be a strong antiviral against BVDV, a cell culture model for HCV, see e.g. Branza-Nichita N, Durantel D, Carrouee-Durantel S, Dwek R A, Zitzmann N., J. Virol. 2001 April; 75(8):3527-36; Durantel, D., et al, J. Virol, 2001, 75, 8987-8998; N. Zitzmann, et al, PNAS, 1999, 96, 11878-11882. Treatment with NB-DNJ leads to decreased infectivity of viral progeny, with less of an effect on the actual number of secreted viruses.

NB-DNJ has been shown to be antiviral against HIV; treatment leads to a relatively small effect on the number of virus particles released from HIV-infected cells, however the amount of infectious virus released is greatly reduced, see e.g. P. B. Fischer, M. Collin, et al (1995), J. Virol 69(9):5791-7; P. B. Fischer, G. B. Karlsson, T. Butters, R. Dwek and F. Platt, J. Virol. 70 (1996a), pp. 7143-7152, P. B. Fischer, G. B. Karlsson, R. Dwek and F. Platt, J. Virol. 70 (1996b), pp. 7153-7160. Clinical trials involving NB-DNJ were conducted in HIV-1 infected patients, and results demonstrated that concentrations necessary for antiviral activity were too high and resulted in serious side-effects in patients, see e.g. Fischl M. A., Resnick L., Coombs R., Kremer A. B., Pottage J. C. Jr, Fass R. J., Fife K. H., Powderly W. G., Collier A. C., Aspinall R. L., et. al., J. Acquir. Immune. Defic. Syndr. 1994 February; 7(2):139-47. No mutant HIV strain resistant to NB-DNJ treatment currently exists.

### ER Protein Folding and Glucosidase I and II

The antiviral effect demonstrated by glucosidase inhibition is thought to be a result of misfolding or retention of viral glycoproteins within the ER, primarily through blocking entry into the calnexin/calreticulin cycle. Following transfer of the triglucosylated oligosaccharide (Glc₃Man₉GlcNAc₂) to an Asn-X-Ser/Thr consensus sequence in the growing polypeptide chain, it is necessary that the three α-linked glucose residues be released before further processing to the mature carbohydrate units can take place. Moreover, the two outer glucose residues must be trimmed to allow entry into the calnexin/calreticulin cycle for proper folding, see e.g. Bergeron, J. J. et. al., Trends Biochem. Sci., 1994, 19, 124-128; Peterson, J. R. et. al., Mol. Biol. Cell, 1995, 6, 1173-1184. The initial processing is affected by an ER-situated integral membrane enzyme with a lumenally-oriented catalytic domain (glucosidase I) that specifically cleaves the α1-2 linked glucose residue; this is followed by the action of glucosidase II, which releases both of the α1-3 linked glucose components.

### Liposomes

Liposomes can deliver water-soluble compounds directly inside the cell, bypassing cellular membranes that act as molecular barriers. The pH sensitive liposome formulation can involve the combination of phosphatidylethanolamine (PE), or its derivatives, such as e.g. DOPE, with compounds containing an acidic group, which act as a stabilizer at neutral pH. Cholesteryl hemisuccinate (CHEMS) can be a good stabilizing molecule as its cholesterol group confers higher stability to the PE-containing vesicles compared to other amphiphilic stabilizers in vivo. The in vivo efficacy of liposome-mediated delivery can depend strongly on interactions with serum components (opsonins) that influence their pharmacokinetics and biodistribution. pH-sensitive liposomes can be rapidly cleared from blood circulation, accumulating in the liver and spleen, however inclusion of lipids with covalently attached polyethylene glycol (PEG) can overcome clearance by the reticuloendothelial system (RES) by stabilizing the net-negative charge on DOPE:CHEMS liposomes, leading to long circulation times. DOPE-CHEMS and DOPE-CHEMS-PEG-PE liposomes and methods of their preparation are described, for example, in V. A. Slepushkin, S. Simoes, P. Dazin, M. S, Newman, L. S. Guo and M. C. P. de Lima, J. Biol. Chem. 272 (1997) 2382-2388; and S. Simoes, V. Slepushkin, P N. Duzgunes and M. C. Pedroso de Lima, Biomembranes 1515 (2001) 23-37, both incorporated herein by reference in their entirety.

Delivery of NB-DNJ encapsulated in DOPE-CHEMS (molar ratio 6:4) is disclosed in US patent application No. US2003/0124160.

### Disclosure

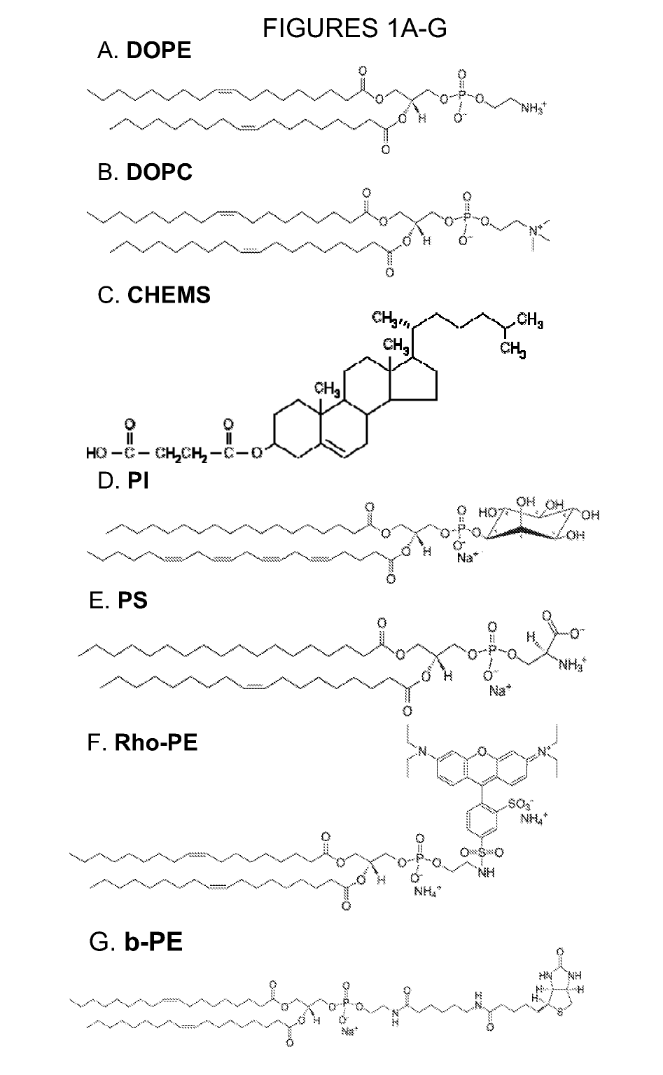

The inventors believe that lipid particles, such as liposomes or micelles, that comprise at least one of PI or PS lipids, see FIG. 1, may be taken efficiently by a cell and fuse with the ER membrane of that cell. The inventors also discovered that the lipid particles, that comprise at least one of PI or PS lipids, can have a high stability in a blood or blood component, such as serum. For example, the liposomes, that comprise at least one of PI or PS lipids, can have a greater stability in serum than DOPE/CHEMS liposomes (molar ratio 6:3) or DOPE/CHEMS/PEG-PE (molar ratio 6:3:0.1) liposomes.

In some embodiments, the lipid particles can contain PI and/or PS lipids at a molar concentration of at least 5% or at least 10% or at least 15% or at least 20% or at least 25% or at least 30% or from 3% to 60% or from 5% to 50% or from 10% to 30%. In some embodiments, a molar concentration of PS lipids in the lipid particle can be at least 5% or at least 10% or at least 15% or at least 20% or at least 25% or at least 30% or from 3% to 60% or from 5% to 50% or from 10% to 30%. In some embodiments, a molar concentration of PI lipids in the lipid particle can be at least 5% or at least 10% or at least 15% or at least 20% or at least 25% or at least 30% or from 3% to 60% or from 5% to 50% or from 10% to 30%. In some embodiments, a combined concentration of PI and PS lipids in the lipid particle can be at least 5% or at least 10% or at least 15% or at least 20% or at least 25% or at least 30% or from 3% to 60% or from 5% to 50% or from 10% to 30%.

The lipid particles may further comprise one or more phopsphatidylethanolamine (PE) lipids or its derivative, such as DOPE. In some embodiments, the PE lipids may comprise PE lipids conjugated with a label, which can be, for example, a fluorophore label, a biotin label or a radioactive label. FIGS. 1A, 1F and 1G present chemical structures of DOPE lipid, Rho-PE lipid, which is an example of a PE lipid conjugated with a fluorophore label, and b-PE lipid, which is an example of PE-lipid conjugated with a biotin label.

In some embodiments, the lipid particles may further comprises at least one of PC or CHEMS liposomes. Yet in some other embodiments, the lipid particles may be such that they do not contain PC and/or CHEMS lipids.

In some embodiments, the lipid particles that comprise PE, PC, PI and PS lipids may be preferred. Such lipid particles may interfere with cellular LDs, which may lead to significantly reduced infectivity of HCV particles secreted from HCV-infected cells treated with these lipid particles. The lipid particles that comprise PE, PC, PI and PS lipids may be used for introducing lipids into HCV-infected cells to interfere with the LD/HCV core protein interaction. Also, the lipid particles that comprise PE, PC, PI and PS lipids may be competing for the same cellular receptors as HCV, therefore out-competing the virus for cellular entry, and reducing viral infectivity.

### Viral Infections

The lipid particles can be used for treating, preventing and/or monitoring a disease or condition caused by or associated with a virus in a subject, which in many cases can be a warm blooded animal such as a mammal or a bird. In many cases, the subject can be a human. In many cases, the disease or condition can be a viral infection. In some embodiments, the lipid particles, that comprise at least one of PI or PS lipids, can be used for treating, preventing and/or monitoring a disease or condition caused by or associated with a virus that belongs to the Flaviviridae family. The Flaviviridae family includes Genus Flavivirus; Genus Hepacivirus and Genus Pestivirus. The Flavivirus Genus includes Gadgets Gully virus (GGYV), Kadam virus (KADV); Kyasanur Forest disease virus (KFDV); Langat virus (LGTV); Omsk hemorrhagic fever virus (OHFV); Powassan virus (POWV); Royal Farm virus (RFV); Tick-borne encephalitis virus (TBEV); Louping ill virus (LIV); Meaban virus (MEAV); Saumarez Reef virus (SREV); Tyuleniy virus (TYUV); Aroa virus (AROAV); Dengue virus (DENV) 1-4; Kedougou virus (KEDV); Cacipacore virus (CPCV); Koutango virus (KOUV); Japanese encephalitis virus (JEV); Murray Valley encephalitis virus (MVEV); St. Louis encephalitis virus (SLEV); Usutu virus (USUV); West Nile virus (WNV); Yaounde virus (YAOV); Kokobera virus (KOKV); Bagaza virus (BAGV); Ilheus virus (ILHV); Israel turkey meningoencephalomyelitis virus (ITV); Ntaya virus (NTAV); Tembusu virus (TMUV); Zika virus (ZIKV); Banzi virus (BANV); Bouboui virus (BOUV); Edge Hill virus (EHV); Jugra virus (JUGV); Saboya virus (SABV); Sepik virus (SEPV); Uganda S virus (UGSV); Wesselsbron virus (WESSV); Yellow fever virus (YFV); Entebbe bat virus (ENTV); Yokose virus (YOKV); Apoi virus (APOIV); Cowbone Ridge virus (CRV); Jutiapa virus (JUTV); Modoc virus (MODV); Sal Vieja virus (SVV); San Perlita virus (SPV); Bukalasa bat virus (BBV); Carey Island virus (CIV); Dakar bat virus (DBV); Montana myotis leukoencephalitis virus (MMLV); Phnom Penh bat virus (PPBV); Rio Bravo virus (RBV). The Hepacivirus Genus includes Hepatitis C virus (HCV, Hep C). The Pestivirus Genus includes Border disease virus; Bovine Diarrhea virus (BVDV); and Classical swine fever virus. The diseases caused by or associated with Flaviviruses include Dengue fever; Japanese encephalitis; Kyasanur Forest disease; Murray Valley encephalitis; St. Louis encephalitis; Tick-borne encephalitis; West Nile encephalitis and Yellow fever. The diseases caused by or associated with Hepaciviruses include Hepatitis C viral infection. The diseases caused by or associated with Pestiviruses include Classical swine fever (CSF) and Bovine Virus Diarrhea (BVD) or Bovine Virus Diarrhea/Mucosal disease (BVD/MD).

In some embodiments, the lipid particles can be used for treating, preventing and/or monitoring a disease or condition caused by or associated with a virus that belongs to the Hepadnaviridae family. The Hepadnaviridae family includes Genus Orthohepadnavirus, which includes Hepatitis B virus and Genus Avihepadnavirus, which includes Duck Hepatitis B virus. The diseases causes by or associated with Hepadnaviruses include Hepatitis B virus infection.

In some embodiments, the lipid particles can be used for treating, preventing and/or monitoring a disease or condition caused or associated with a virus that belongs to the Retroviridae family. The Retroviridae family includes Genus Alpharetrovirus, which includes Avian leukosis virus; Genus Betaretrovirus, which includes Mouse mammary tumour virus; Genus Gammaretrovirus, which includes Murine leukemia virus and Feline leukemia virus; Genus Deltaretrovirus, which includes Bovine leukemia virus and Human T-lymphotropic virus; Genus Epsilonretrovirus, which includes Walleye dermal sarcoma virus; Genus Lentivirus, which includes Human immunodeficiency virus 1, Simian immunodeficiency virus and Feline immunodeficiency virus; Genus Spumavirus, which includes Chimpanzee foamy virus. The diseases and conditions caused by or associated with viruses belonging to the Retroviridae family include HIV 1 infection.

In some embodiments, the lipid particles can be used for treating, preventing and/or monitoring a disease or condition caused by or associated with a glycoprotein containing virus. The lipid particles may be used for treatment and prevention of an infection, such as a viral infection, when administered as a part of a composition to a subject, such as human. In some embodiments, such an infection may be an infection caused or associated with a glycoprotein containing virus, i.e. a virus that contains at least one glycoprotein. Yet in some embodiments, such an infection may be a hepatitis infection, such as HCV infection or HBV infection. Yet in some embodiments, such an infection may be a retroviral infection such as HIV. Yet in some embodiment, the infection may be a flaviriral infection, such as HCV.

When the lipid particle is used for treating an HIV infection, it may reduce the infectivity of HIV particles secreted from HIV-infected cells. When the lipid particle is used for treating an HCV infection, it may interfere with cellular LDs and reduce the infectivity of HCV particles secreted from HCV infected cells. Lipid particles that include PE, PC, PI and PS lipids may be preferred in such a case.

Although the present inventions are limited by the theory of their operation, the inventors believe that the lipid particles that include PE, PC, PI and PS lipids may compete for the same cellular receptors as HCV, therefore out-competing the virus cellular entry and reducing viral infectivity.

### Active Agent

In some embodiments, at least one agent, such as a therapeutic agent or an imaging agent, may be encapsulated inside the lipid particle. Such an agent may be, for example, a water soluble molecule, a peptide or an amino acid. The composition comprising the lipid particle with the encapsulated active agent can be used for treating, preventing or monitoring a disease or condition, for which the active agent is known to be effective. The disease or condition may be any disease or condition for which intracellular delivery of the active agent may be beneficial.

The use of lipid particles, that contain PI and/PS lipids, may allow for delivery of the encapsulated material into the ER lumen of a cell.

In some embodiments, the agent encapsulated inside the lipid particle can be, an α-glucosidase inhibitor. In some embodiments, the α-glucosidase inhibitor can be ER α-glucosidase inhibitor, which may be ER α-glucosidase I inhibitor or ER α-glucosidase II inhibitor. In general, any virus that relies on interactions with calnexin and/or calreticulin for proper folding of its viral envelope glycoproteins, can be targeted with ER α-glucosidase inhibitor.

The alpha-glucosidase inhibitor can be an agent that inhibits host alpha-glucosidase enzymatic activity by at least about 10%, at least about 15%, at least about 20%, at least about 25%, at least about 30%, at least about 35%, at least about 40%, at least about 50%, at least about 60%, at least about 70%, at least about 80%, or at least about 90%, or more, compared to the enzymatic activity of the alpha-glucosidase in the absence of the agent. The term “alpha-glucosidase inhibitor” encompasses both naturally occurring and synthetic agents that inhibit host alpha-glucosidase activity. Suitable alpha-glucosidase inhibitors include, but not limited to, deoxynojirimycin and N-substituted deoxynojirimycins, such as compounds of Formula I and pharmaceutically acceptable salts thereof:

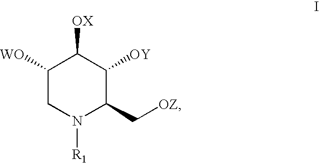

where R₁ is selected from substituted or unsubstituted alkyl groups, which can be branched or straight chain alkyl group; substituted or unsubstituted cycloalkyl groups; substituted or unsubstituted aryl groups, substituted or unsubstituted oxaalkyl groups, substituted or unsubstituted arylalkyl, cycloalkylalkyl, and where W, X, Y, and Z are each independently selected from hydrogen, alkanoyl groups, aroyl groups, and haloalkanoyl groups.

In some embodiments, R₁ can be selected from C1-C20 alkyl groups or C3-C12 alkyl groups. In some embodiments, R₁ can be selected from ethyl, propyl, isopropyl, butyl, isobutyl, tert-butyl, pentyl, neopentyl, isopentyl, n-hexyl, heptyl, n-octyl, n-nonyl and n-decyl. In some embodiments, R₁ can be butyl or nonyl.

In some embodiments, R₁ can be an oxalkyl, which can be C1-C20 alkyl groups or C3-C12 alkyl group, which can also contain 1 to 5 or 1 to 3 or 1 to 2 oxygen atoms. Examples of oxalkyl groups include —(CH₂)₂—O—(CH₂)₅CH₃, —(CH₂)₂—O—(CH₂)₆CH₃, —(CH₂)₆OCH₂CH₃, and —(CH₂)₂OCH₂CH₂CH₃.

In some embodiments, R₁ can be an arylalkyl group. Examples of arylalkyl groups include C1-C12-Ph groups, such as C3-Ph, C4-Ph, C5-Ph, C6-Ph and C7-Ph. In some embodiments, the compound of Formula I can be selected from, but is not limited to N-(n-hexyl-)-1,5-dideoxy-1,5-imino-D-glucitol; N-(n-heptyl-)-1,5-dideoxy-1,5-imino-D-glucitol; N-(n-octyl-)-1,5-dideoxy-1,5-imino-D-glucitol; N-(n-octyl-)-1,5-dideoxy-1,5-imino-D-glucitol, tetrabutyrate; N-(n-nonyl-)-1,5-dideoxy-1,5-imino-D-glucitol, tetrabutyrate; N-(n-decyl-)-1,5-dideoxy-1,5-imino-D-glucitol, tetrabutyrate; N-(n-undecyl-)-1,5-dideoxy-1,5-imino-D-glucitol, tetrabutyrate; N-(n-nonyl-)-1,5-dideoxy-1,5-imino-D-glucitol; N-(n-decyl-)-1,5-dideoxy-1,5-imino-D-glucitol; N-(n-undecyl-)-1,5-dideoxy-1,5-imino-D-glucitol; N-(n-dodecyl-)-1,5-dideoxy-1,5-imino-D-glucitol; N-(2-ethylhexyl)-1,5-dideoxy-1,5-imino-D-glucitol; N-(4-ethylhexyl)-1,5-dideoxy-1,5-imino-D-glucitol; N-(5-methylhexyl)-1,5-dideoxy-1,5-imino-D-glucitol; N-(3-propylhexyl)-1,5-dideoxy-1,5-imino-D-glucitol; N-(1-pentylpentylhexyl)-1,5-dideoxy-1,5-imino-D-glucitol; N-(1-butylbutyl)-1,5-dideoxy-1,5-imino-D-glucitol; N-(7-methyloctyl-)-1,5-dideoxy-1,5-imino-D-glucitol; N-(8-methylnonyl)-1,5-dideoxy-1,5-imino-D-glucitol; N-(9-methyldecyl)-1,5-dideoxy-1,5-imino-D-glucitol; N-(10-methylundecyl)-1,5-dideoxy-1,5-imino-D-glucitol; N-(6-cyclohexylhexyl-)-1,5-dideoxy-1,5-imino-D-glucitol; N-(4-cyclohexylbutyl)-1,5-dideoxy-1,5-imino-D-glucitol; N-(2-cyclohexylethyl)-1,5-dideoxy-1,5-imino-D-glucitol; N-(1-cyclohexylmethyl)-1,5-dideoxy-1,5-imino-D-glucitol; N-(1-phenylmethyl)-1,5-dideoxy-1,5-imino-D-glucitol; N-(3-phenylpropyl)-1,5-dideoxy-1,5-imino-D-glucitol; N-(3-(4-methyl)-phenylpropyl)-1,5-dideoxy-1,5-imino-D-glucitol; N-(6-phenylhexyl)-1,5-dideoxy-1,5-imino-D-glucitol; N-(n-nonyl-)-1,5-dideoxy-1,5-imino-D-glucitol, tetrabutyrate; N-(n-decyl-)-1,5-dideoxy-1,5-imino-D-glucitol, tetrabutyrate; N-(n-undecyl-)-1,5-dideoxy-1,5-imino-D-glucitol, tetrabutyrate; N-(n-dodecyl-)-1,5-dideoxy-1,5-imino-D-glucitol, tetrabutyrate; N-(2-ethylhexyl)-1,5-dideoxy-1,5-imino-D-glucitol, tetrabutyrate; N-(4-ethylhexyl)-1,5-dideoxy-1,5-imino-D-glucitol, tetrabutyrate; N-(5-methylhexyl)-1,5-dideoxy-1,5-imino-D-glucitol, tetrabutyrate; N-(3-propylhexyl)-1,5-dideoxy-1,5-imino-D-glucitol, tetrabutyrate; N-(1-pentylpentylhexyl)-1,5-dideoxy-1,5-imino-D-glucitol, tetrabutyrate; N-(1-butylbutyl)-1,5-dideoxy-1,5-imino-D-glucitol, tetrabutyrate; N-(7-methyloctyl-)-1,5-dideoxy-1,5-imino-D-glucitol, tetrabutyrate; N-(8-methylnonyl)-1,5-dideoxy-1,5-imino-D-glucitol, tetrabutyrate; N-(9-methyldecyl)-1,5-dideoxy-1,5-imino-D-glucitol, tetrabutyrate; N-(10-methylundecyl)-1,5-dideoxy-1,5-imino-D-glucitol, tetrabutyrate; N-(6-cyclohexylhexyl-)-1,5-dideoxy-1,5-imino-D-glucitol, tetrabutyrate; N-(4-cyclohexylbutyl)-1,5-dideoxy-1,5-imino-D-glucitol, tetrabutyrate; N-(2-cyclohexylethyl)-1,5-dideoxy-1,5-imino-D-glucitol, tetrabutyrate; N-(1-cyclohexylmethyl)-1,5-dideoxy-1,5-imino-D-glucitol, tetrabutyrate; N-(1-phenylmethyl)-1,5-dideoxy-1,5-imino-D-glucitol, tetrabutyrate; N-(3-phenylpropyl)-1,5-dideoxy-1,5-imino-D-glucitol, tetrabutyrate; N-(3-(4-methyl)-phenylpropyl)-1,5-dideoxy-1,5-imino-D-glucitol, tetrabutyrate; N-(6-phenylhexyl)-1,5-dideoxy-1,5-imino-D-glucitol, tetrabutyrate; pharmaceutically acceptable salts thereof; and mixtures of any two or more thereof.

Diseases and conditions, for which N-substituted deoxynojirimycins can be effective, are disclosed in U.S. Pat. Nos. 4,849,430; 4,876,268; 5,411,970; 5,472,969; 5,643,888; 6,225,325; 6,465,487; 6,465,488; 6,515,028; 6,689,759; 6,809,083; 6,583,158; 6,589,964; 6,599,919; 6,916,829; 7,141,582. The diseases and conditions, for which N-substituted deoxynojirimycins can be effective, include, but not limited to HIV infection; Hepatitis infections, including Hepatitis C and Hepatitis B infections; lysosomal lipid storage diseases including Tay-Sachs disease, Gaucher disease, Krabbe disease and Fabry disease; and cystic fibrosis. In some embodiments, the α-glucosidase inhibitor can be N-oxaalkylated deoxynojirimycins or N-alkyloxy deoxynojirimycin, such as N-hydroxyethyl DNJ (Miglitol or Glyset™) described in U.S. Pat. No. 4,639,436.

In some embodiments, the α-glucosidase inhibitor can be a castanospermines and/or a castanospermine derivative, such as a compounds of Formula (I) and pharmaceutically acceptable salts thereof disclosed in US patent application no. 2006/0194835, including 6-O-butanoyl castanospermine (celgosivir), and compounds and pharmaceutically acceptable salt thereof of Formula II disclosed in PCT publication no. WO01054692.

Diseases and conditions, for which castanospermine and its derivatives can be effective, are disclosed, in U.S. Pat. Nos. 4,792,558; 4,837,237; 4,925,796; 4,952,585; 5,004,746; 5,214,050; 5,264,356; 5,385,911; 5,643,888; 5,691,346; 5,750,648; 5,837,709; 5,908,867; 6,136,820; 6,583,158; 6,589,964; 6,656,912 and U.S. publications 20020006909; 20020188011; 20060093577; 20060194835; 20080131398. The diseases and conditions, for which castanospermine and its derivatives can be effective, include, but not limited, retroviral infections including HIV infection; celebral malaria; hepatitis infections including Hepatitis B and Hepatitis C infections; diabetes and lysosomal storage disorders. In some embodiments, the alpha glucosidase inhibitor can be acarbose (0-4,6-dideoxy-4-[[(1S,4R,5 S,6S)-4,5,6-trihydroxy-3-(hydroxymethyl)-2-cyc-lohexen-1-yl]amino]-α-D-glucopyranosyl-(1→4)—O-→D-gluc-opyranosyl-(1→4)-D-glucose), or Precose®. Acarbose is disclosed in U.S. Pat. No. 4,904,769. In some embodiments, the alpha glucosidase inhibitor can be a highly purified form of acarbose (see, e.g., U.S. Pat. No. 4,904,769).

In some embodiments, the agent encapsulated inside the liposome can be an ion channel inhibitor. In some embodiments, the ion channel inhibitor can be an agent inhibiting the activity of HCV p7 protein. Ion channel inhibitors and methods of identifying them are detailed in US patent publication 2004/0110795. Suitable ion channel inhibitors include compounds of Formula I and pharmaceutically acceptable salts thereof, including N-(7-oxa-nonyl)-1,5,6-trideoxy-1,5-imino-D-galactitol (N-7-oxa-nonyl 6-MeDGJ or UT231B) and N-10-oxaundecul-6-MeDGJ. Suitable ion channel inhibitors also include, but not limited to, N-nonyl deoxynojirimycin, N-nonyl deoxynogalactonojirimycin and N-oxanonyl deoxynogalactonojirimycin.

In some embodiments, the agent encapsulated inside the liposome can be an iminosugar. Suitable iminosugars include both naturally occurring iminosugars and synthetic iminosugars.

In some embodiments, the iminosugar can be deoxynojirimycin or N-substituted deoxynojirimycin derivative. Examples of suitable N-substituted deoxynojirimycin derivatives include, but not limited to, compounds of Formula II of the present application, compounds of Formula I of U.S. Pat. No. 6,545,021 and N-oxaalkylated deoxynojirimycins, such as N-hydroxyethyl DNJ (Miglitol or Glyset®) described in U.S. Pat. No. 4,639,436.

In some embodiments, the iminosugar can be castanospermine or castanospermine derivative. Suitable castanospemine derivatives include, but not limited to, compounds of Formula (I) and pharmaceutically acceptable salts thereof disclosed in US patent application No. 2006/0194835 and compounds and pharmaceutically acceptable salt thereof of Formula II disclosed in PCT publication No. WO01054692. In some embodiments, the iminosugar can be deoxynogalactojirimycin or N-substituted derivative thereof such as those disclosed in PCT publications No. WO99/24401 and WO01/10429. Examples of suitable N-substituted deoxynogalactojirimycin derivatives include, but not limited to, N-alkylated deoxynogalactojirimycins (N-alkyl-1,5-dideoxy-1,5-imino-D-galactitols), such as N-nonyl deoxynogalactojirimycin, and N-oxa-alkylated deoxynogalactojirimycins (N-oxa-alkyl-1,5-dideoxy-1,5-imino-D-galactitols), such as N-7-oxanonyl deoxynogalactojirimycin.

In some embodiments, the iminosugar can be N-substituted 1,5,6-trideoxy-1,5-imino-D-galactitol (N-substituted MeDGJ) including, but not limited to compounds of Formula II:

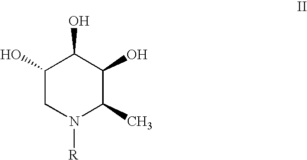

wherein R is selected from substituted or unsubstituted alkyl groups, substituted or unsubstituted cycloalkyl groups, substituted or unsubstituted heterocyclyl groups, or substituted or unsubstituted oxaalkyl groups. In some embodiments, substituted or unsubstituted alkyl groups and/or substituted or unsubstituted oxaalkyl groups comprise from 1 to 16 carbon atoms, or from 4 to 12 carbon atoms or from 8 to 10 carbon atoms. In some embodiments, substituted or unsubstituted alkyl groups and/or substituted or unsubstituted oxaalkyl groups comprise from 1 to 4 oxygen atoms, and from 1 to 2 oxygen atoms in other embodiments. In other embodiments, substituted or unsubstituted alkyl groups and/or substituted or unsubstituted oxaalkyl groups comprise from 1 to 16 carbon atoms and from 1 to 4 oxygen atoms. Thus, in some embodiments, R is selected from, but is not limited to —(CH₂)₆OCH₃, —(CH₂)₆OCH₂CH₃, —(CH₂)₆O(CH₂)₂CH₃, —(CH₂)₆O(CH₂)₃CH₃, —(CH₂)₂O(CH₂)₅CH₃, —(CH₂)₂O(CH₂)₆CH₃, and —(CH₂)₂O(CH₂)₇CH₃. N-substituted MeDGJs are disclosed, for example, in PCT publication No. WO01/10429.

In some embodiments, the agent encapsulated inside the liposome can include a nitrogen containing compound having formula III or a pharmaceutically acceptable salt thereof:

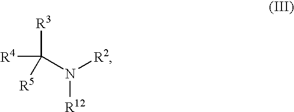

wherein R¹² is an alkyl such as C₁-C₂₀, or C₁-C₆ or C₇-C₁₂ or C₈-C₁₆ and can also contain from 1 to 5 or from 1 to 3 or from 1 to 2 oxygen, R¹² can be an oxa-substituted alkyl derivative. Examples if oxa-substituted alkyl derivatives include 3-oxanonyl, 3-oxadecyl, 7-oxanonyl and 7-oxadecyl.

R² is hydrogen, R³ is carboxy, or a C₁-C₄ alkoxycarbonyl, or R² and R³, together are

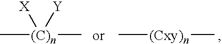

e wherein n is 3 or 4, each X, independently, is hydrogen, hydroxy, amino, carboxy, a C₁-C₄ alkylcarboxy, a C₁-C₄ alkyl, a C₁-C₄ alkoxy, a C₁-C₄ hydroxyalkyl, a C₁-C₆ acyloxy, or an aroyloxy, and each Y, independently, is hydrogen, hydroxy, amino, carboxy, a C₁-C₄ alkylcarboxy, a C₁-C₄ alkyl, a C₁-C₄ alkoxy, a C₁-C₄ hydroxyalkyl, a C₁-C₆ acyloxy, an aroyloxy, or deleted (i.e. not present);

R⁴ is hydrogen or deleted (i.e. not present); and

R⁵ is hydrogen, hydroxy, amino, a substituted amino, carboxy, an alkoxycarbonyl, an aminocarbonyl, an alkyl, an aryl, an aralkyl, an alkoxy, a hydroxyalkyl, an acyloxy, or an aroyloxy, or R³ and R⁵, together, form a phenyl and R⁴ is deleted (i.e. not present).

In some embodiments, the nitrogen containing compound has the formula:

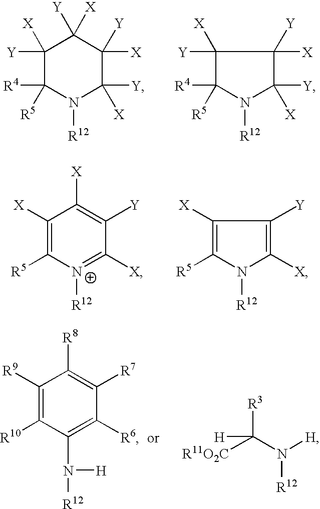

where each of R⁶-R¹⁰, independently, is selected from the group consisting of hydrogen, hydroxy, amino, carboxy, C₁-C₄ alkylcarboxy, C₁-C₄ alkyl, C₁-C₄ alkoxy, C₁-C₄ hydroxyalkyl, C₁-C₄ acyloxy, and aroyloxy; and R¹¹ is hydrogen or C₁-C₆ alkyl. The nitrogen-containing compound can be N-alkylated piperidine, N-oxa-alkylated piperidine, N-alkylated pyrrolidine, N-oxa-alkylated pyrrolidine, N-alkylated phenylamine, N-oxa-alkylated phenylamine, N-alkylated pyridine, N-oxa-alkylated pyridine, N-alkylated pyrrole, N-oxa-alkylated pyrrole, N-alkylated amino acid, or N-oxa-alkylated amino acid. In certain embodiments, the N-alkylated piperidine, N-oxa-alkylated piperidine, N-alkylated pyrrolidine, or N-oxa-alkylated pyrrolidine compound can be an iminosugar. For example, in some embodiments, the nitrogen-containing compound can be N-alkyl-1,5-dideoxy-1,5-imino-D-galactitol (N-alkyl-DGJ) or N-oxa-alkyl-1,5-dideoxy-1,5-imino-D-galactitol (N-oxa-alkyl-DGJ) having the formula:

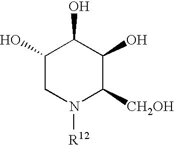

or N-alkyl-1,5,6-trideoxy-1,5-imino-D-galactitol (N-alkyl-MeDGJ) or N-oxa-alkyl-1,5,6-trideoxy-1,5-imino-D-galactitol having (N-oxa-alkyl-MeDGJ) having the formula:

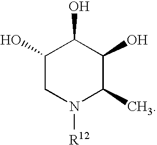

As used herein, the groups have the following characteristics, unless the number of carbon atoms is specified otherwise. Alkyl groups have from 1 to 20 carbon atoms and are linear or branched, substituted or unsubstituted. Alkoxy groups have from 1 to 16 carbon atoms, and are linear or branched, substituted or unsubstituted. Alkoxycarbonyl groups are ester groups having from 2 to 16 carbon atoms. Alkenyloxy groups have from 2 to 16 carbon atoms, from 1 to 6 double bonds, and are linear or branched, substituted or unsubstituted. Alkynyloxy groups have from 2 to 16 carbon atoms, from 1 to 3 triple bonds, and are linear or branched, substituted or unsubstituted. Aryl groups have from 6 to 14 carbon atoms (e.g., phenyl groups) and are substituted or unsubstituted. Aralkyloxy (e.g., benzyloxy) and aroyloxy (e.g., benzoyloxy) groups have from 7 to 15 carbon atoms and are substituted or unsubstituted. Amino groups can be primary, secondary, tertiary, or quaternary amino groups (i.e., substituted amino groups). Aminocarbonyl groups are amido groups (e.g., substituted amido groups) having from 1 to 32 carbon atoms. Substituted groups can include a substituent selected from the group consisting of halogen, hydroxy, C₁₋₁₀ alkyl, C₂₋₁₀ alkenyl, C₁₀ acyl, or C₁₋₁₀ alkoxy.

The N-alkylated amino acid can be an N-alkylated naturally occurring amino acid, such as an N-alkylated a-amino acid. A naturally occurring amino acid is one of the 20 common α-amino acids (Gly, Ala, Val, Leu, Ile, Ser, Thr, Asp, Asn, Lys, Glu, Gln, Arg, His, Phe, Cys, Trp, Tyr, Met, and Pro), and other amino acids that are natural products, such as norleucine, ethylglycine, ornithine, methylbutenyl-methylthreonine, and phenylglycine. Examples of amino acid side chains (e.g., R⁵) include H (glycine), methyl (alanine), —CH₂C(O)NH₂ (asparagine), —CH₂—SH (cysteine), and —CH(OH)CH₃ (threonine).

An N-alkylated compound can be prepared by reductive alkylation of an amino (or imino) compound. For example, the amino or imino compound can be exposed to an aldehyde, along with a reducing agent (e.g., sodium cyanoborohydride) to N-alkylate the amine. Similarly, a N-oxa-alkylated compound can be prepared by reductive alkylation of an amino (or imino) compound. For example, the amino or imino compound can be exposed to an oxa-aldehyde, along with a reducing agent (e.g., sodium cyanoborohydride) to N-oxa-alkylate the amine.

The nitrogen-containing compound can include one or more protecting groups. Various protecting groups are well known. In general, the species of protecting group is not critical, provided that it is stable to the conditions of any subsequent reaction(s) on other positions of the compound and can be removed at the appropriate point without adversely affecting the remainder of the molecule. In addition, a protecting group may be substituted for another after substantive synthetic transformations are complete. Clearly, where a compound differs from a compound disclosed herein only in that one or more protecting groups of the disclosed compound has been substituted with a different protecting group, that compound is within the invention. Further examples and conditions are found in Greene, Protective Groups in Organic Chemistry, (1^{st} Ed., 1981, Greene & Wuts, 2^{nd} Ed., 1991).

The nitrogen-containing compound can be purified, for example, by crystallization or chromatographic methods. The compound can be prepared stereospecifically using a stereospecific amino or imino compound as a starting material.

The amino and imino compounds used as starting materials in the preparation of the long chain N-alkylated compounds are commercially available (Sigma, St. Louis, Mo.; Cambridge Research Biochemicals, Norwich, Cheshire, United Kingdom; Toronto Research Chemicals, Ontario, Canada) or can be prepared by known synthetic methods. For example, the compounds can be N-alkylated imino sugar compounds or oxa-substituted derivatives thereof. The imino sugar can be, for example, deoxygalactonojirmycin (DGJ), 1-methyl-deoxygalactonojirimycin (MeDGJ), deoxynorjirimycin (DNJ), altrostatin, 2R,5R-dihydroxymethyl-3R,4R-dihydroxypyrrolidine (DMDP), or derivatives, enantiomers, or stereoisomers thereof.

In some embodiments, the agent encapsulated inside the lipid particle can be a compound of Formula IV or V:

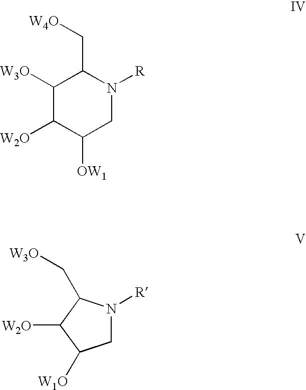

wherein R is:

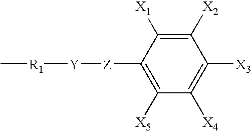

### R′ is:

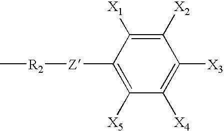

R₁ is a substituted or unsubstituted alkyl group; R₂ is a substituted or unsubstituted alkyl group; W₁₋₄ are independently selected from hydrogen, substituted or unsubstituted alkyl groups, substituted or unsubstituted haloalkyl groups, substituted or unsubstituted alkanoyl groups, substituted or unsubstituted aroyl groups, or substituted or unsubstituted haloalkanoyl groups; X₁₋₅ are independently selected from H, NO₂, N₃, or NH₂; Y is absent or is a substituted or unsubstituted C₁-alkyl group, other than carbonyl; Z is selected from a bond or NH; provided that when Z is a bond, Y is absent, and provided that when Z is NH, Y is a substituted or unsubstituted C₁-alkyl group, other than carbonyl; and Z′ is a bond or NH. Compounds of formula IV and V and methods of their synthesis are disclosed, for example, in U.S. publication No. US2007/0275998. Non-limiting examples of compounds of Formula IV and V include N—(N′-{4′azido-2′-nitrophenyl)-6-aminohexyl)-deoxynojirimycin (NAP-DNJ) and N—(N′-{2,4-dinitrophenyl)-6-aminohexyl)-deoxynojirimycin (NDP-DNJ). The syntheses of a variety of iminosugar compounds have been described. For example, methods of synthesizing DNJ derivatives are known and are described, for example, in U.S. Pat. Nos. 5,622,972, 5,401,645, 5,200,523, 5,043,273, 4,994,572, 4,246,345, 4,266,025, 4,405,714, and 4,806,650. Methods of synthesizing other iminosugar derivatives are known and are described, for example, in U.S. Pat. Nos. 4,861,892, 4,894,388, 4,910,310, 4,996,329, 5,011,929, 5,013,842, 5,017,704, 5,580,884, 5,286,877, and 5,100,797 and PCT publication No. WO 01/10429. The enantiospecific synthesis of 2R,5R-dihydroxymethyl-3R,4R-dihydroxypyrrolidine (DMDP) is described by Fleet & Smith (Tetrahedron Lett. 26:1469-1472, 1985). The imaging agent can be a tagged or fluorescent aqueous material, such as calcein, or fluorescently labeled molecules such as siRNA, antibodies, or other small molecule inhibitors. Tagged lipophilic material can also be incorporated into lipid particles for incorporation into cellular membranes, such as the rh-PE lipid used for visualizing liposomes in cells and other similar lipids with tags for visualization or purification. This can also include tagged lipophilic proteins or drugs with fluorescent moieties or other tags for visualization or purification.

### Targeting Moieties

In some embodiments, the composition comprising the lipid particle may comprise at least one targeting moiety, which can be conjugated with the lipid particle or intercalated into a lipid layer or bilayer of the particle. In some embodiments, the targeting moiety may be a ligand, which may be a ligand of an envelop protein of a virus, or an antibody, which may be an antibody against an envelop protein of a virus. Such a moiety may used for targeting the particle to a cell infected with the virus. Such targeting moiety may be also used for achieving sterilizing immunity against a viral infection associated with or caused by the virus.

In some embodiments, the targeting moiety may comprise with a gp120/gp41 targeting moiety. In such a case, the composition comprising the lipid particle may be preferred for treating and/or preventing an HIV-1 infection. The gp120/gp41 targeting moiety can comprise a sCD4 molecule or a monoclonal antibody, such as IgG 2F5 or IgG b12 antibodies.

In some embodiments, the targeting moiety can comprise E1 or E2 targeting moiety, such as E1 or E2 proteins from HCV. In such a case, the composition comprising the lipid particle may be preferred for treating and/or preventing an HCV infection. In some cases, targeting moiety may be also a molecule that can target E I and/or E2 proteins, such as specific antibodies to these proteins, and soluble portions of cell receptors, such as a soluble CD81 or SR-BI molecules.

### Intercalated Moieties

In some embodiments, the lipid particle may comprise one or more moieties intercalated into its lipid layer or bilayer. Examples of intercalated moieties include, but not limited, to a transmembrane protein, a protein lipid conjugate, a labeled lipid, a lipophilic compound or any combination thereof.

In some embodiments, the intercalated moiety may include a lipid-PEG conjugate. Such a conjugate may increase the in vivo stability of the lipid particle and/or increase its circulation time.

In some embodiments, the intercalated moiety may include a long alkyl chain iminosugar, such as C₇-C16 alkyl or oxaalkyl substituted N-deoxynojrimycin (DNJ) or C7-C16 alkyl or oxaalkyl substituted deoxygalactonojirimycin (DGJ). Non-limiting examples of long alkyl chain iminosugars include N-nonyl DNJ and N-nonyl DGJ.

In some embodiments, the intercalated moiety may include a fluorophore-lipid conjugate, which may be used for labeling the ER membrane of a cell contacted with the lipid bilayer particle. Such labeling may be useful for live and/or fixed-cell imaging in eukaryotic cells.

The use of lipid particles, that comprise PI and/or PS lipids, may result in delivery of the intercalated moiety into the ER membrane of a cell.

### Polyunsaturated Lipid Particles

The present inventors also believe that lipid particles, such as liposomes, that include at least one polyunsaturated lipid may be effective in treating and/or preventing infections, such as a viral infection, in a subject, such as a human.

In some embodiments, the polyunsaturated lipids may constitute at least 5% by mole or at least 10% by mole or at least 15% by mole or at least 20% by mole or at least 25% by mole or at least 30% by mole or at least 35% by mole or at least 40% by mole or at least 45% by mole or at least 50% by mole or at least 55% by mole or at least 60% by mole or at least 65% by mole or at least 70% by mole or at least 75% by mole or at least 80% by mole or at least 85% by mole or least 90% by mole or at least 95% by mole of the total lipids of the lipid particle.

As used herein, the term “polyunsaturated lipid” refers to a lipid that contains more than one unsaturated chemical bond, such as a double or a triple bond, in its hydrophobic tail.

In some embodiments, the polyunsaturated lipid can have from 2 to 8 or from 3 to 7 or from 4 to 6 double bonds in its hydrophobic tail.

As used herein, the term “polyunsaturated lipid particle” refers to a lipid particle that comprises at least one polyunsaturated lipid.

In some embodiments, the lipid particle may include more than one polyunsaturated lipid.

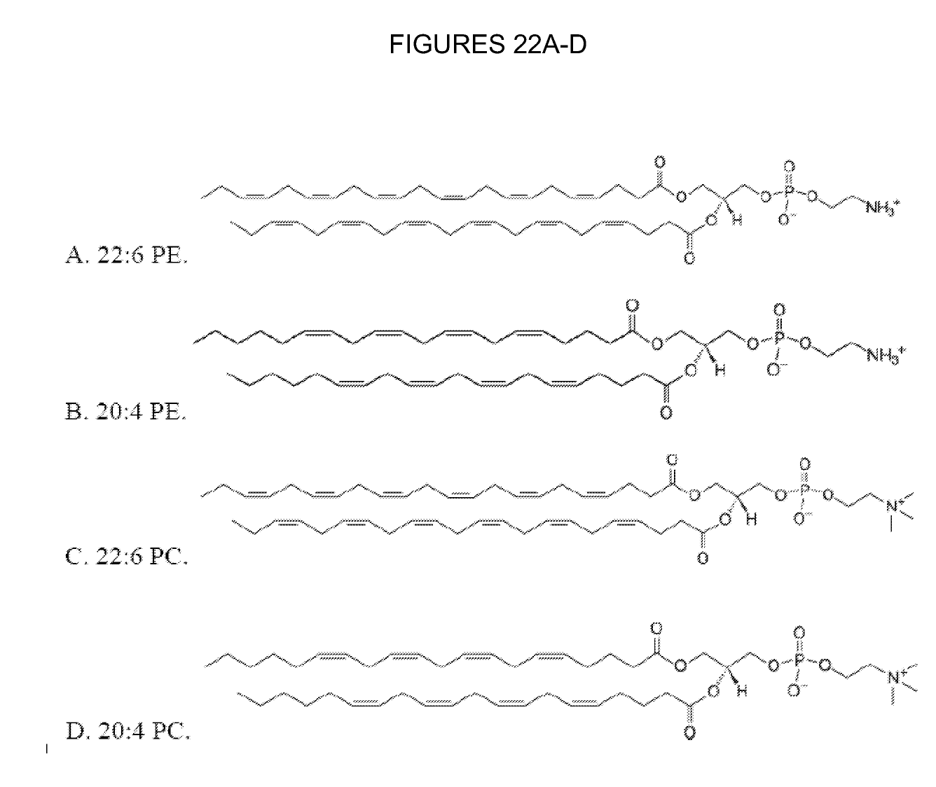

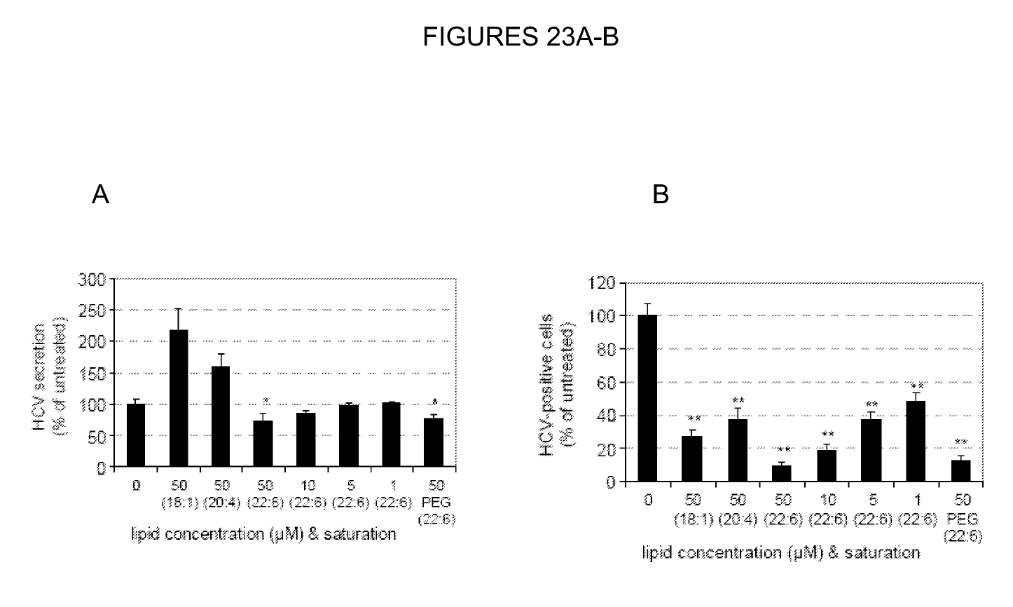

Preferably, the polyunsaturated lipid particle contains at least one of polyunsaturated PE or polyunsaturated PC lipids. FIGS. 22 A-D provides chemical structures of exemplary polyunsaturated PE and PC lipids.

The lipid particle may further include one or more additional lipids such as PI, PS, or CHEMS.

The polyunsaturated lipid particle that includes at least one of polyunsaturated PE or polyunsaturated PC lipids may be used for treating, preventing monitoring a disease or condition caused by or associated with a virus, such as the diseases or conditions disclosed above. In many embodiments, such a disease or condition can be a viral infection. In some embodiments, such an infection may be a hepatitis infection, such as an HCV infection or an HBV infection. Yet in some embodiments, such an infection may be a retroviral infection, such as HIV. Yet in some embodiment, the infection may be a flaviriral infection, such as an HCV infection.

In some embodiments, the polyunsaturated lipid particle may encapsulate at least one active agent, such as the agents disclosed above.

In some embodiments, the polyunsaturated lipid particle may comprise at least one moiety intercalated into a lipid layer or bilayer of the particle, which may be any of the intercalated moieties disclosed above.

In some embodiments, a composition that includes the lipid particle may include a targeting moiety associated with the particle, which again may be any of the targeting moieties disclosed above.

In some embodiments, the polyunsaturated lipid particle comprising PE, PC, PI and PS lipids, at least one of which is unsaturated, may be preferred for treating or preventing HCV infection. Although the present inventions are not limited by their theory of operation, the inventors believe that the polyunsaturated lipid particle comprising PE, PC, PI and PS lipids can significantly decrease the secretion of HCV virions from HCV-infected cells because the delivery of polyunsaturated lipids to the site of HCV replication, which is the ER membrane, can reduce HCV RNA replication and subsequently HCV secretion.

### Administering

In some embodiments, the composition comprising the lipid particles can be administered to a cell. The cell can be a cell infected with a virus. In many cases, the contacted cell can be a cell from a warm blooded animal such as a mammal or a bird. In some embodiments, the contacted cell can be a cell from a human. In some embodiments, the composition comprising the lipid particles administering the composition to an individual. The subject can be a warm blooded animal, such as a mammal or a bird. In many cases, the subject can be a human. In some embodiments, the composition comprising the lipid particles can be administered by intravenous injection. Yet in some embodiments, the composition comprising the lipid particles can be administered via a parenteral routes other than intravenous injection, such as intraperitoneal, subcutaneous, intradermal, intraepidermal, intramuscular or transdermal route. Yet in some embodiments, the composition comprising the lipid particles can be administered via a mucosal surface, e.g. an ocular, intranasal, pulmonary, intestinal, rectal and urinary tract surfaces. Administration routes for lipid containing compositions, such as liposomal compositions, are disclosed, for example, in A. S. Ulrich, Biophysical Aspects of Using Liposomes as Delivery Vehicles, Bioscience Reports, Volume 22, Issue 2, April 2002, 129-150.

Delivery of a therapeutic agent, such as NB-DNJ, via the lipid particles, such as liposomes into the ER lumen can lower an effective amount of the therapeutic agent required for inhibition of ER-glucosidase compared to non-liposome methods. For example, for NB-DNJ, the IC90 can be reduced by at least 100, or by at least 500, or by at least 1000, or by at least 5000, or by at least 10000, or by at least 50000 or by at least 100000. Such a reduction of the effective antiviral amount of NB-DNJ can result in final concentrations of administered NB-DNJ that are one or more orders of magnitude below toxic levels in mammals, in particular, humans.

In some cases, the composition comprising the lipid particles comprising a therapeutic agent, such as NB-DNJ, can be contacted with the infected cell in combination with one or more additional therapeutic agents, such as antiviral agents. In some cases, such additional therapeutic agents can be co-encapsulated with NB-DNJ into the lipid particle. Yet in some cases, contacting the infected cell with such additional therapeutic agents can be a result of administering the additional therapeutic agents to a subject comprising the cell. The administration of the additional therapeutic agents can be carried out by adding the therapeutic agents to the composition. Yet in some cases, the administration of the additional therapeutic agents can be performed separately from administering the composition comprising the lipid particles containing NB-DNJ. Such separate administration can be performed via an administration pathway that can the same or different that the administration pathway used for the composition comprising the lipid particles.

Combination therapy may not only reduce the effective dose of an agent required for antiviral activity, thereby reducing its toxicity, but may also improve the absolute antiviral effect as a result of attacking the virus through multiple mechanisms.

In addition, combination therapy can provide means for circumventing or decreasing a chance of development of viral resistance.

The particular additional therapeutic agent(s) that can be used in combination the liposome containing NB-DNJ can depend of the disease or condition being treated. For example, for a hepatitis infection, such as HBV, HCV or BVDV infection, such therapeutic agent(s) can be a nucleoside or nucleotide antiviral agent and/or an immunostimulating/immunomodulating agent. Various nucleoside agents, nucleotide agents and immunostimulating/immunomodulating agents that can be used in combination with NB-DNJ for treatment of hepatitis are exemplified in U.S. Pat. No. 6,689,759 issued Feb. 10, 2004, to Jacob et. al. For example, for treatment of hepatitis C infection, NB-DNJ can be encapsulated in the liposome in combination with 1-b-D-ribofuranosyl-1H-1,2,4-triazole-3-carboxamide (ribavirin), as a nucleoside agent, and interferon such as interferon alpha, as an immunostimulating/immunomodulating agent. The treatment of hepatitis infections with ribavirin and/or interferon is discussed, for example, in U.S. Pat. Nos. 6,172,046; 6,177,074; 6,299,872; 6,387,365; 6,472,373; 6,524,570 and 6,824,768.

For treating an HIV infection, a therapeutic agent that can be used in combination with a liposome containing NB-DNJ can be an anti-HIV agent, which can be, for example, nucleoside Reverse Transcriptase (RT) inhibitor, such as (−)-2′-deoxy-3′-thiocytidine-5′-triphosphate (3TC); (−)-cis-5-fluoro-1-[2-(hydroxy-methyl)-[1,3-oxathiolan-5-yl]cytosine (FTC); 3′-azido-3′-deoxythymidine (AZT) and dideoxy-inosine (ddI); a non-nucleoside RT inhibitors, such as N11-cyclopropyl-4-methyl-5,11-dihydro-6H-dipyrido[3,2-b:2′3′-e]-[1,4]diazepin-6-one (Neviparine), a protease inhibitor or a combination thereof. Anti HIV therapeutic agents can be used in double or triple combinations, such as AZT, DDI, and nevirapin combination. In some embodiments, the agent encapsulated inside the lipid particle may be, for example, an agent disclosed on pages 14-20 of U.S. patent application Ser. No. 11/832,891, which is incorporated herein by reference in its entirety. The lipid particle may deliver the encapsulated agent inside the lumen of the ER upon fusion of lipids of the lipid particle with the ER membrane.

### Labeled Lipids

In some embodiments, the lipid particle may include at least one labeled lipid, that is labeled with at least one label such as a radioactive label, a fluorophore label or a biotin label, thus, making the particle itself labeled.

The labeled lipid particles may be used for specific labeling an ER membrane of a cell, which can be later imaged. A type of cells that can be imaged by this technology is not particularly limited. The imaging may be performed by, for example, live or fixed imaging. The fixed imaging can refer to imaging of dead cells that may be fixed with a fixing medium such as paraformaldehyde. Cells can be permeabilized and probed with antibodies to detect specific proteins or labels prior to mounting and imaging. For live-cell microscopy, cells can be still alive in media while the imaging is taking place.

The labeled particle may be used for labeling a virus. Examples of viruses, which may be labeled using such an approach, include ER-budding viruses, such as BVDV and HCV. When the label is a fluorophore label, the labeled lipid bilayer particle may be used for imaging of the labeled virus, which can be live and/or fixed imaging. When the label is a biotin label, the labeled lipid particle may be used for purification of the labeled viral particles. In some cases, such purification can be performed using streptavidin. Streptavidin can be linked to sepharose beads for batch purification of biotin-labeled material.

The invention is further illustrated by, though in no way limited to, the following examples.

### Example

### 1. Liposome Preparation

Liposomes were prepared fresh for all assays described. Chloroform solutions of lipids were placed into glass tubes and the solvent was evaporated under a stream of nitrogen gas. Unless stated otherwise, lipid films were hydrated by vortexing in 1×PBS buffer to a final lipid concentration of 5 mM. The resulting multilamellar vesicles were extruded 11 times through a polycarbonate filter of 100 nm pore diameter using a Mini-Extruder device. Liposomes were filter sterilized using a 0.22 μm filter unit. FIG. 1(A)-(E) presents lipids used in these studies: A. DOPE; B. DOPC; C. CHEMS; D. PI; E. PS. PEG-PE used in the experiments was PEG (MW-2000)-distearoylphospatidylethanolamine. All lipids except cholesteryl hemisuccinate were purchased from Avanti Polar Lipids (USA), as were all the materials for preparation of liposomes. Cholesteryl hemisuccinate was purchased from Sigma (UK).

### 2. Liposomes Containing PI and/or PS Localize to the ER

The purpose of this experiment was to treat Huh7.5 cells (human liver cells) with liposomes containing PE and PC or CHEMS, with or without PI and/or PS lipids, to determine their co-localization with the ER membrane. Liposomes were labeled by incorporation of a rhodamine-tagged PE (Rh-PE) into all liposomes. The ER membrane of Huh7.5 cells was labeled using an anti-EDEM antibody. EDEM antibody was purchased from Santa Cruz Biotechnology (USA). Co-localization was determined by confocal microscopy. Significant co-localization can serve as a proof of liposomes fusing with the ER membrane of Huh7.5 cells.

### 2.1 Specific Methodology for Visualizing Liposome Co-Localization with the ER Membrane of Liposome-Treated Huh7.5 Cells:

Liposomes with the lipid composition PE:CH, PE:PC, PE:CH:PI, PE:PC:PI, PE:CH:PS, PE:PC:PS, PE:CH:PI:PS, and PE:PC:PI:PS were prepared as previously described and included 1% (total moles) of Rh-PE for visualization. Huh7.5 cells were allowed to adhere overnight onto number 1.5 glass cover slides before media was exchanged and replaced with fresh media containing liposomes added to a final lipid concentration of 50 μM. After a 5 min incubation at 37° C./5% CO₂, media containing liposomes were removed and cells were washed twice with 1×PBS, and incubated in fresh media for an additional 30 min before being fixed in 4% paraformaldehyde diluted in 1×PBS/0.1% Tween-20 for 15 min, and washed twice in 1×PBS/0.1% Tween-20. Cells were then incubated for 1 h in 1×PBS/0.1% Tween-20 containing 4 μg/ml anti-EDEM antibody, washed twice in 1×PBS/0.1% Tween-20, incubated 1 h in 1×PBS/0.1% Tween-20 containing 4 μg/ml FITC-labeled secondary antibody, and washed twice more. Cells were stained with DAPI prior to mounting onto microscope slides. Confocal images were taken using a Carl Zeiss LSM microscope, and image analysis was done using the LSM software v5.10. FIG. 1F shows a structure of the Rh-PE lipid used in these assays:

### 2.2 Co-Localization of Different Liposomes with the Er Marker EDEM in Huh7.5 Cells

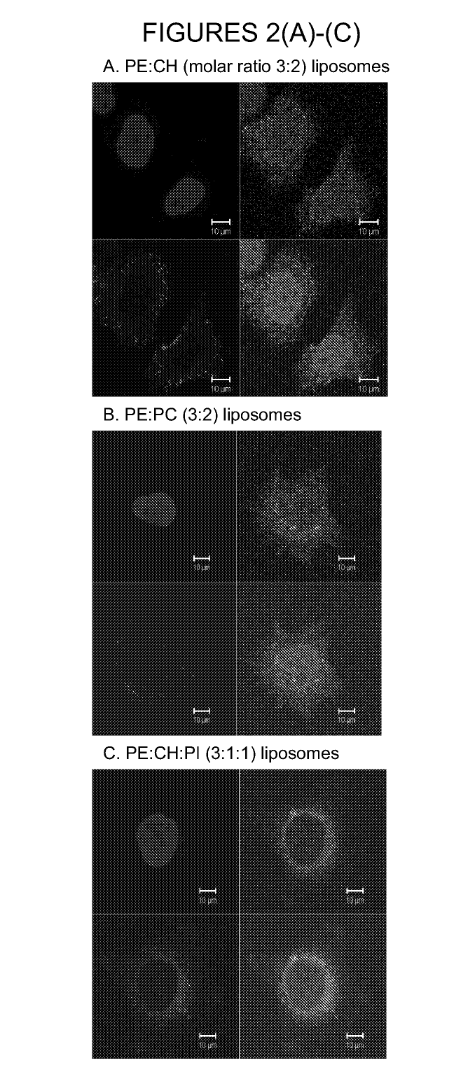

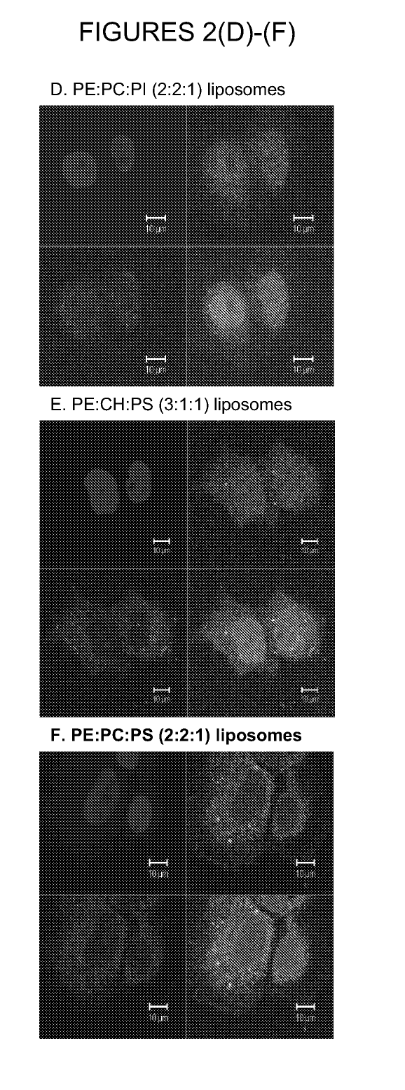

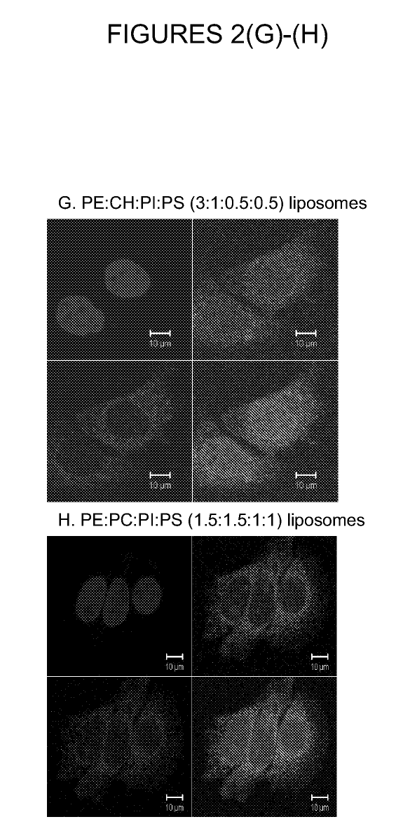

FIG. 2(A)-(F) demonstrate that liposomes containing the lipids PI and/or PS co-localized with the ER-membrane protein EDEM. Liposomes were incubated with Huh7.5 cells for 5 min before media was changed and cells were incubated in liposome-free media. Cells were fixed and probed with an anti-EDEM antibody (green, top right image) following a 30 min incubation, and co-localization with the Rh-PE lipids from liposomes (red, bottom left images) was determined by confocal microscopy. DAPI (blue, top left images) is used as a nuclear stain. Co-localization was measured by the presence of yellow within the merged images (bottom right). Experiments were repeated three times, and representative images are shown. FIG. 2A. PE:CH (molar ratio 3:2) liposomes; FIG. 2B. PE:PC (3:2) liposomes; FIG. 2C. PE:CH:PI (3:1:1) liposomes; FIG. 2D. PE:PC:PI (2:2:1) liposomes; FIG. 2E. PE:CH:PS (3:1:1) liposomes; FIG. 2F. PE:PC:PS (2:2:1) liposomes; FIG. 2G. PE:CH:PI:PS (3:1:0.5:0.5) liposomes; FIG. 2H. PE:PC:PI:PS (1.5:1.5:1:1) liposomes

The co-localization of liposomes with an ER membrane marker (EDEM) was quantified using images obtained as described above.

### 2.3. Methodology for Quantification of Image Co-Localization

Percentage co-localization was measured using MetaMorph software (v.7, Molecular Devices, Downingtown, Pa., U.S.A.). Images were filtered using a median filter set to 3×3 pixels, and thresholds used to determine integrated co-localization between two images (rh-PE/red images and EDEM/green images) were set at the mean intensity plus 1 standard deviation (SD) for each. Reported values represent the mean±SD of 30 cells.

### 2.4. Results of Percent Co-Localization Analysis of Liposomes with the ER Marker (EDEM)

Results of the quantification of image co-localization can suggest that incorporation of 20% PI or 20% PS into DOPE:CH or DOPE:DOPC liposomes significantly increases co-localization with the ER membrane. Liposomes composed of DOPE:CH:PI and DOPE:CH:PS demonstrated 52% (SD=8.0%) and 46% (SD=8.1%) co-localization, respectively, compared to 13% (SD=6.6%) for DOPE:CH alone. Similarly, compositions of DOPE:DOPC:PI and DOPE:DOPC:PS demonstrated 64% (SD=8.1%) and 48% (SD=7.6%) co-localization, respectively, compared to 12% (SD=4.7%) for DOPE:DOPC liposomes. The combination of 20% PI and 20% PS within DOPE:CH and DOPE:DOPC liposomes further increased co-localization to the ER membrane such that DOPE:CH:PI:PS liposomes demonstrated 76% (SD=8.7%) ER membrane co-localization and 88% (SD=3.5%) co-localization was observed with DOPE:DOPC:PI:PS liposomes.

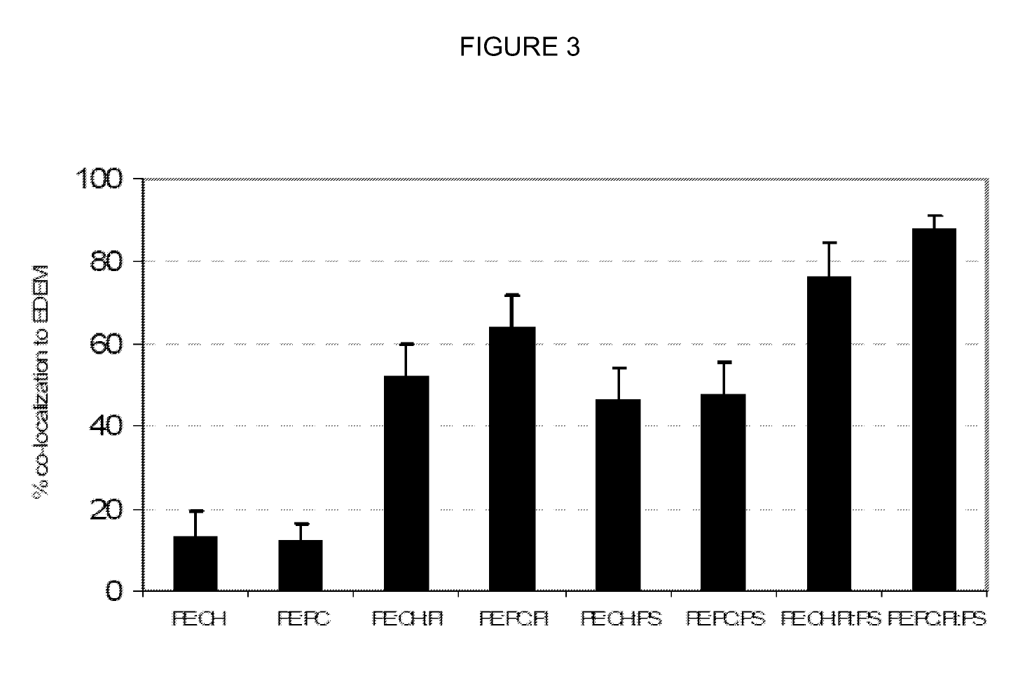

FIG. 3 shows calculated co-localization of liposome-delivered rh-DOPE with the EDEM antibody was determined by analyzing 30 individual cells per liposome preparation using MetaMorph software, where the thresholds used for determining % co-localization were set to the mean intensity plus one SD for each image. Results shown represent the mean co-localization and SD for the 30 cells.

These results can demonstrate that only liposomes containing the lipids PI and/or PS in combination with PE show increased co-localization with the ER marker in Huh7.5 cells following a 5 min pulse with liposomes and a 30 min chase. Since ER liposomes, i.e. liposomes that contain PI and/or PS lipids, demonstrate significant co-localization with the ER marker, fluorescent-labeled ER liposomes may be used as a quick and inexpensive technology for labeling the ER membrane in eukaryotic cells.

### 3. Lipids Delivered Via ER Liposomes are Incorporated into the Envelope of Viruses Known to Assemble and Bud from the ER Membrane

The purpose of the following experiment was to treat Madin-Darby bovine kidney (MDBK) cells infected with bovine viral diarrhea virus (BVDV) and HCV cell culture (HCVcc)-infected Huh7.5 cells with liposomes shown to co-localize with the ER membrane by confocal microscopy and look for the incorporation of tagged liposome lipids within secreted viral particles. BVDV and HCV are both viruses that assemble and bud from the ER membrane; therefore incorporation of tagged lipids delivered via liposomes into secreted viral particles suggests fusion of liposomes with the ER membrane of these cells. HIV-1-infected peripheral blood mononuclear cells (PBMCs) are used as a control in order to detect the incorporation of lipids into viruses that bud from the plasma membrane.

### 3.1. Specific Methodology for Monitoring the Incorporation of Liposome Lipids into Secreted Viral Particles Using a Biotinylated PE Lipid

BVDV cell culture: Madin Darby bovine kidney cells (MDBK) cells were seeded at 3×10⁵ cells/well of a 6-well plate in complete DMEM/10% FBS, infected with ncp BVDV strain Pe515 (National Animal Disease Laboratory, United Kingdom) at a multiplicity of infection (MOI) of 0.1, and passaged into 2 ml of fresh RPMI 1640 medium containing 10% (vol/vol) fetal calf serum at a 1:8 dilution every 3 days. Liposome treatments were begun after a stable infection was achieved, as determined by RT-RCR to quantify secreted BVDV particles. Quantitative PCR was performed on 500 μl of supernatant using the QIAamp Viral RNA Purification Kit (QIAGEN), following the manufacturers' protocol. Real-time PCR was done using a SyBr Green Mix (QIAGEN) and primers directed against the ncp BVDV RNA (forward: TAG GGC AAA CCA TCT GGA AG, reverse primer: ACT TGG AGC TAC AGG CCT CA).

JC-1 HCV cell culture (HCVcc): Huh7.5 cells (Apath, LLC, Saint Louis, U.S.A.) were grown in complete DMEM (100 U/ml penicillin, 100 μg/ml streptomycin, 2 mM L-glutamine, and 1×MEM) with 10% fetal bovine serum (FBS). All incubations were at 37° C./5% CO₂. Cells were infected for 1 h at MOI=0.5 and liposome treatments were started when over 50% of cells tested positive for HCVcc infection, as determined by HCV core protein immunofluorescence. The quantification of viral RNA from supernatant, as well as the infectivity of secreted particles was determined using quantitative PCR and core protein immunofluorescence, respectively.

HIV cell culture: Peripheral blood mononuclear cells (PBMCs) from four uninfected donors were isolated using Histopaque density gradient centrifugation (Sigma-Aldrich, Gillingham, U.K.), pooled, and stimulated with phytohemagglutinin (PHA, 5 μg/ml) for 48 h followed by interleukin-2 (IL2, 40 U/ml) for 72 h in complete RPMI (RPMI plus 10% FBS, 100 U/ml penicillin, 100 μg/ml streptomycin, and 2 mM L-glutamine) before starting experiments. All incubations were at 37° C./5% CO₂, unless stated otherwise. To infect cells, 4×10⁶ PHA-activated PBMCs and 100 TCID₅₀ (tissue culture infectious dose 50%) of primary isolate stock were incubated together in 2 ml complete RPMI/10% FBS per well in a 6-well plate. Cells were infected for 16 h, and were washed three times with complete RPMI medium before commencing incubations with liposomes.

Purification of secreted biotin-labeled particles: Virus-infected cells were grown in a 75 cm² flask before the medium was replaced with medium containing 50 μM b-PE-labeled 22:6 ER liposomes and left to incubate 48 h. Cells were then washed twice in PBS and incubated in fresh medium without liposomes for a further 24 h.

Supernatant containing secreted particles was harvested, cells were counted using trypan blue staining, and the supernatants were standardized to sample cell numbers using PBS. Secreted HCVcc and BVDV were titered by quantitative PCR, and the infectivity of secreted virions was determined. HIV-1 was quantified by p24 capture ELISA. High performance streptavidin sepharose (GE Healthcare) was used to capture biotinylated particles. Sepharose beads were washed twice by diluting 1:50 (vol:vol) in PBS, gently mixing at room temperature for 5 min, and pelleted with centrifugation for 3 min at 1500 rpm. Sepharose was resuspended to form a 50% slurry in PBS and added to culture supernatant (200 μl 50% slurry per 10 ml culture supernatant). Sepharose and supernatant were left to incubate 1 h at room temperature with gentle rocking, before sepharose beads were washed five times in PBS as described above. To quantify the amount of b-PE-labeled virions, 1 ml of culture supernatant was put aside, 500 μl of which was used for total virus quantification, and 500 μl were captured on streptavidin sepharose, washed five times in PBS, and used directly for RNA quantification by incubating beads with viral RNA lysis buffer (QIAGEN) for HCVcc and BVDV RT-PCR analysis, or by incubating in 1% empigen for p24 HIV ELISA assays.

### 3.2. Incorporation of b-PE Lipids into Secreted Viral Particles

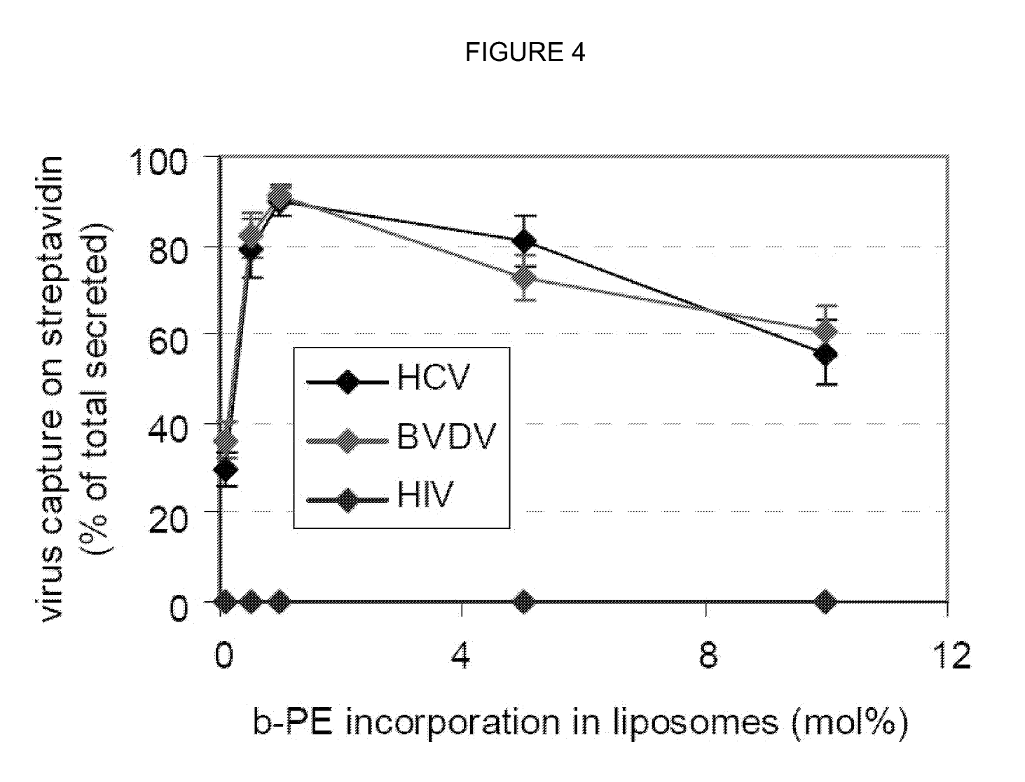

FIG. 1G shows a structure of the biotinylated PE lipid (b-PE) used. Biotin-labeled PE (b-PE) was incorporated into ER liposomes, i.e. liposomes that contain PI and/or PS lipids, at 0.1, 0.5, 1, 5, or 10 mol %, and the optimal concentration for tagging secreted HCV and BVDV (two ER-budding viruses) was determined to be 1%, capturing 90% (SD=3.6%) and 91% (SD=1.5%) of the total number of secreted virions, respectively (FIG. 4). PBMCs infected with a primary isolate of HIV-1 (LAI) were also treated with b-ER liposomes, and none of the secreted HIV-1 particles contained detectable amounts of the tagged lipid (FIG. 4). This result can highlight the specificity of this system for delivering lipids to the ER and ER-associated membranes, as productive HIV-1 assembly occurs at the plasma membrane

FIG. 4 shows results of experiments for ER liposomes (final lipid concentration of 50 μM) containing b-PE lipids incubated with JC-1-infected Huh7.5 cells, BVDV-infected MDBK cells, or HIV-1-infected PBMCs for 48 h. Infected cells were washed, and b-PE-labeled viral particles secreted during a subsequent 24 h incubation period in the absence of liposomes were captured using streptavidin-sepharose resin. Results are displayed as the percentage of tagged viral particles captured by streptavidin in relation to the total amount of secreted virions within the same sample (100%).

Results in FIG. 4 can demonstrate that lipids delivered to BVDV-infected MDBK cells and HCVcc-infected Huh7.5 cells via ER-localizing liposomes (liposomes comprising PE in combination with PI and/or PS) are present in the majority of BVDV and HCVcc viral envelopes, but not in HIV envelopes, secreted during liposome treatment. Because BVDV and HCV are known to assemble and bud from the ER membrane, whereas HIV assembles at and buds from the plasma membrane, this is further evidence that liposomes containing PI and/or PS lipids are capable of fusion with the ER membrane of cells.

The incorporation of a tagged lipid into ER-budding viruses following treatment with ER liposomes may be not limited to biotinylated lipids, but fluorescent lipids may also be used to produce virions containing a fluorescent lipid for visualization by fluorescence microscopy.

### 3.3. Methodology for Imaging Rh-PE-Tagged HCVcc Following Rh-PE Liposome Treatment Using Confocal Microscopy

Huh7.5 cells were grown to full confluency in a 75 cm² flask before medium was replaced with medium containing 50 μM rh-PE-labeled 22:6 ER liposomes. Cells were left to incubate for 48 h, washed twice in PBS, and were then incubated in fresh medium without liposomes for 24 h. Supernatants containing secreted particles were harvested. Secreted HCVcc was titered by quantitative PCR, and the infectivity of secreted virions was determined as previously described. For visualization of rh-HCVcc by confocal microscopy naïve Huh7.5 cells were allowed to adhere overnight onto number 1.5 glass cover slides in complete DMEM/10% FCS before the medium was replaced with rh-HCVcc viral stock and incubated 1 h. Following the infection, cells were washed twice with PBS, and fresh medium was replaced for various incubation times, washed twice with 1×PBS, fixed in methanol:acetone (1:1, vol:vol) for 10 min, and finally washed twice in 1×PBS/0.1% Tween-20. Cells were then incubated for 1 h in 1×PBS/0.1% Tween-20 containing a primary antibody, washed four times in 1×PBS/0.1% Tween-20, incubated 1 h in 1×PBS/0.1% Tween-20 containing a fluorescent-labeled secondary antibody, and washed four times more. Cells were stained with DAPI prior to mounting onto microscope slides. Confocal images were taken using a Carl Zeiss LSM microscope, and image analysis was done using the LSM software v5.10.

### 3.4. Visualization of Rh-PE-Tagged HCVcc by Confocal Microscopy

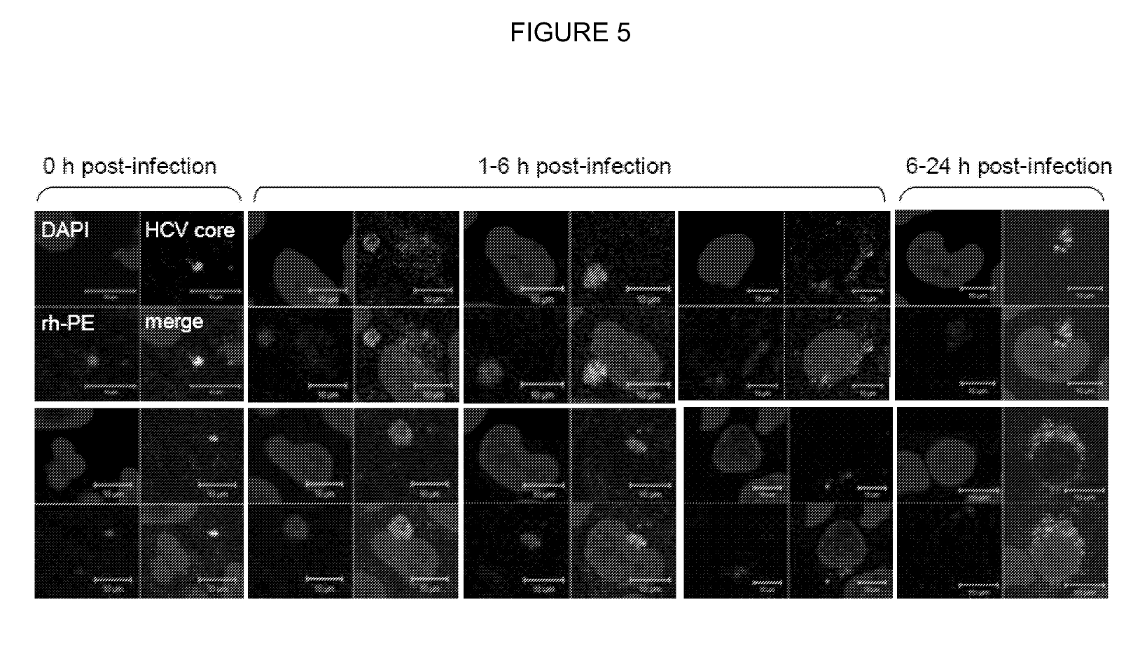

Using 1% rh-ER liposomes and fixed-cell confocal microscopy we have visualized an HCVcc infection in Huh7.5 cells (FIG. 5). Rh-tagged HCVcc was collected for 24 h following a 48 h incubation in the presence of 1% rh-ER liposomes, and used to infect naïve cells at a MOI=0.1 for 1 h. Fixed confocal images were taken immediately following the 1 h infection, as well as 6 h and 24 h post-infection and permeabilized cells were probed with an anti-HCV core antibody to positively identify HCVcc particles. In these images the core-positive particles appear as a single cluster of approximately 1 μm in diameter on the surface of cells up until 1 h post-infection, at which point this cluster appears to become endocytosed and diffuses into a cluster of approximately 5 μm. This large cluster moves towards the nucleus of the cells, forming a characteristic indent of the nucleus of infected cells (FIG. 5), at which point the cluster disperses and rh-tagged lipids begin to separate from HCV core protein. Increased levels of core protein are observed in cells approximately 24 h post-infection, and may represent an established infection and de novo core protein synthesis.

FIG. 5 shows results of experiments for Rh-PE-tagged JC-1 HCVcc (red, bottom-left panels) incubated with naïve Huh7.5 cells for 1 h (MOI=0.1), following which cells were washed and incubated for a further 0, 6, or 24 h in fresh media. After each incubation time, cells were fixed and stained with an anti-HCV core antibody (green, top-right panel) and DAPI (blue, top-left panel) prior to mounting onto microscope slides and confocal microscopy imaging. Merged images are shown in the bottom-right panels. Representative images from each incubation period are shown. Although fixed-cell confocal microscopy was used in these analyses, this technology offers a method for labeling virions with a wide selection of lipid-fluorophore conjugates for tracking by live-cell microscopy. This type of incorporation technology is not limited to biotin or fluorescent tagged lipids, as other lipid conjugates or transmembrane proteins can also be incorporated into ER liposomes for specific delivery to the ER membranes of cells.

### 4. Lipids Delivered Via ER Liposomes have a Longer Lifetime in the Cell Compared to pH-Sensitive Liposomes

The purpose of this experiment was to treat MDBK cells with fluorescent-labeled liposomes to monitor there uptake and incorporation into cellular membranes over time. pH-sensitive liposomes, i.e. DOPE-CHEMS or DOPE-CHEMS-PEG-PE liposomes, which do not contain PI and PS lipids, can be thought to enter cells and, following disruption of the liposome membrane in endosomes, lipids are thought continue along the endosomal pathway to the lysosome. If liposomes, that contain PI and/or PS lipids, are capable of fusion with other membranes within the cell they should have a longer lifetime compared to pH-sensitive liposomes. Rho-PE lipids delivered to cells via liposomes were visualized by a fluorescent microscope over a period of 48 hours following a 5 min treatment with MDBK cells.

### 4.1. Specific Methodology for Monitoring Liposome Incorporation into Cellular Membranes

PE:CH, PE:CH:PI, and PE:CH:PS liposomes were prepared as previously described and included 1% (total moles) of Rh-PE for visualization. MDBK cells were seeded onto 6 well plates at 50% confluency and left to adhere overnight. Cells were washed twice in 1×PBS followed by treatment with Rh-labeled liposomes added to 2 ml of complete RPMI to a final lipid concentration of 50 μM for 5 min at 37° C., 5% CO₂. After the 5 min incubation, cells were washed twice in 1×PBS, 2 ml of fresh complete RPMI medium was added to each well, and plates were left to incubate for 1, 10, 24, and 48 h. At the end of each incubation time, cells were washed twice before being fixed in 4% paraformaldehyde diluted in 1×PBS/0.1% Tween-20 for 15 min, and washed twice in 1×PBS/0.1% Tween-20. Cells were stained with DAPI prior to imaging. Fluorescent images were taken using a Nikon Eclipse TE2000-U microscope, and image analysis was done using the Nikon ACT-1 software v2.70.

### 4.2. Incorporation of Liposomes into Cellular Membranes

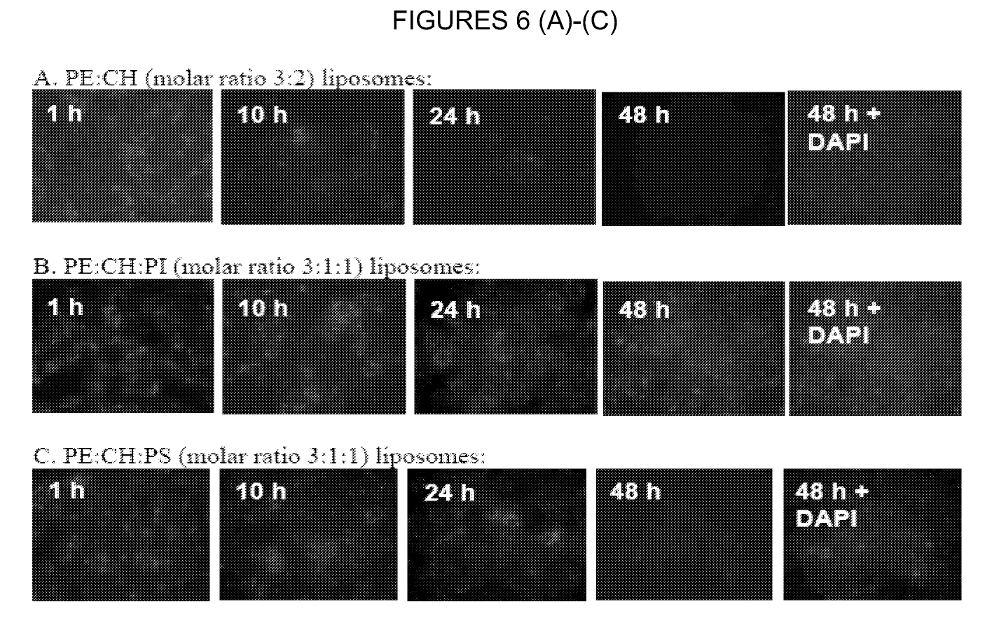

FIGS. 6(A)-(C) shows fluorescent microscope images of liposomes composed of the lipids PE in combination with PI or PS demonstrate increased incorporation into cellular membranes compared to pH-sensitive liposomes. MDBK cells were treated with Rh-PE labeled liposomes for 5 min before cells were washed and left to incubate in media only for 1, 10, 24, and 48 h. Following each incubation time, cells were fixed and Rh-PE lipids (red) are visualized under a fluorescent microscope. DAPI (blue) is used as a nuclear stain. Experiment was repeated twice and representative images from one experiment are shown. FIG. A. PE:CH (molar ratio 3:2) liposomes. FIG. 6 B. PE:CH:PI (molar ratio 3:1:1) liposomes. FIG. 6C. PE:CH:PS (molar ratio 3:1:1) liposomes.

Results in FIG. 6 show that liposomes composed of PE in combination with PI or PS are capable of incorporation into the membranes of MDBK cells. While Rh-PE lipids delivered to cells via PE:CH lipids almost disappear 24 h following the removal of liposomes from the cellular media, lipids delivered via PE:CH:PI and PE:CH:PC are still present in cells for over 48 h, suggesting greater incorporation into membranes.

### 4.3. Quantifying Liposome Uptake and Lipid Retention in Treated Cells

To monitor the rate of liposome uptake in Huh7.5 cells over a 4 day incubation period, cells were incubated with DOPE:CH and DOPE:DOPC:PI:PS liposomes containing 1% rh-PE with a final lipid concentration of 50 μM in medium. Cells were seeded at low density, and liposome uptake was measured in relation to cell growth. Following the 4 day incubation, treated Huh7.5 cells were washed and returned to medium without any liposomes to monitor the half-life of rh-DOPE lipids delivered via DOPE:CH and DOPE:DOPC:PI:PS liposomes.

### 4.4. Methodology for Quantifying Liposome Uptake and Lipid Retention in Treated Cells

Liposomes were prepared as previously described and included 1% (total moles) of rh-PE for monitoring their uptake in cells. For long-term (4 day) liposome uptake assays Huh7.5 cells were seeded onto 6 well plates at 10⁵ cells/well in 2 ml of complete DMEM medium/10% FBS. Rh-PE-labeled liposomes were added to cells to a final phospholipid concentration of 50 μM and left to incubate at 37° C./5% CO₂ for 2, 24, 48, 72, and 96 h. Following incubation times, cells were harvested and analyzed. For analysis, cells were washed twice in 1×PBS, counted, resuspended in 200 μl 1×PBS/0.5% Triton X-100, and transferred to a 96 well plate to read in a spectrofluorometer at λex=550 nm, λem=590 nm. To measure the retention of rh-PE lipids inside Huh7.5 cells following the 96 h incubation described above, cells were washed three times in 1×PBS, media were replaced with fresh DMEM/10% FBS, and cells were left to incubate for a further 8, 24, 30, and 48 h. Following incubation times, cells were harvested and analyzed as described above.

### 4.5. Results of Liposome Uptake and Lipid Retention Assays in Huh7.5 Cells

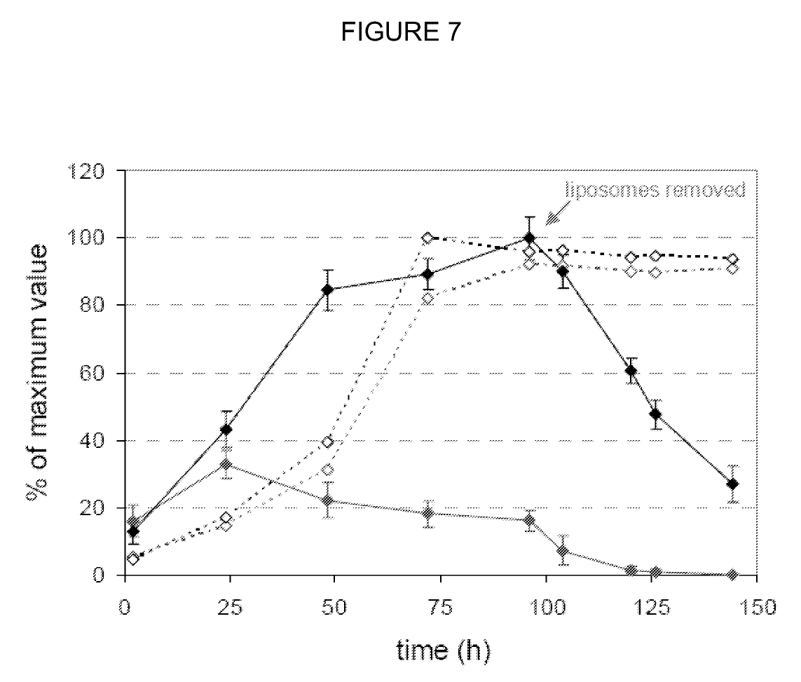

As shown in FIG. 7, actively dividing Huh7.5 cells demonstrated continuous uptake of DOPE:DOPC:PI:PS liposomes over the 4 day incubation period. At day 4, DOPE:DOPC:PI:PS-treated cells demonstrated a fluorescence of 1.5×10-3 AU/cell (SD=3.4×10-4 AU/cell), which is 6-fold greater than that observed with DOPE:CH liposome treatment (2.5×10-4 AU/cell (SD=5.5×10-5 AU/cell)). In fact, the maximum fluorescence observed in DOPE:CH-treated cells was reached following only a 24 h treatment period (5.0×10-4 AU/cell (SD=1.1×10-4)), after which cell-associated fluorescence slowly decreases suggesting either decreased liposome uptake or increased efflux of rh-PE lipids, or both.

Based on these experiments, rh-DOPE lipids from DOPE:CH liposomes demonstrated a half-life in cells of approximately 7 h following removal of liposomes from the medium. In the case of cells treated with DOPE:DOPC:PI:PS liposomes, the rh-DOPE half-life was extended to approximately 29 h, suggesting greater incorporation of these liposomes into the membranes of treated cells.

FIG. 7 shows results of experiments for ER-targeting liposomes that demonstrate increased cellular uptake and lipid retention inside Huh7.5 cells. Rh-labeled liposomes (50 μM final lipid concentration) were incubated with Huh7.5 cells for 4 days (96 hours). Liposome uptake into cells was monitored throughout the incubation period and is presented as the fluorescence observed per cell for both DOPE:CH (red, solid line) and DOPE:DOPC:PI:PS liposomes (black, solid line) in relation to the maximum value (1.5×10⁻³ AU/cell, DOPE:DOPC:PI:PS liposomes, 96 h reading). Fluorescence was measured at λex=550 nm, λem=590 nm. Following the 96 h incubation, cells were washed and placed into fresh media (without liposomes) to monitor the retention of rh-DOPE lipids within cells over a further 48 h. Cell growth during the incubation period is presented for both DOPE:CH (red, dotted line) and DOPE:DOPC:PI:PS liposomes (black, dotted line) in relation to the maximum value (2.4×10⁶ cells/ml, DOPE:DOPC:PI:PS liposomes, 72 h reading). Data represent the mean and SD of triplicate samples from three independent experiments.

### 5. ER Liposomes Demonstrate Increased Stability and Cellular Uptake in the Presence of Serum

The general use of liposomes as a drug delivery system has been hindered by several problems. Among these is the leakage of liposomal contents mediated by serum proteins. Calcein-encapsulating liposomes was used to monitor the stability of liposomes in cell-free medium containing 10% FBS over a 4 day period. Calcein is a water-soluble, self-quenching fluorophore that will remain quenched when encapsulated inside liposomes; however, liposome destabilization will induce leakage and subsequent dequenching of the fluorescence.

### 5.1. Methodology for Quantifying Liposome Stability and Cellular Uptake in FBS

To monitor the stability of liposomes in the presence of 10% FBS, calcein-loaded liposomes were prepared, separated from unencapsulated calcein by size-exclusion chromatography, and added to complete DMEM/10% FBS in the absence of cells, final phospholipid concentration of 50 μM. Liposomes were left to incubate for 4 days, and every 24 h a sample of liposome-containing medium was taken to monitor calcein dequenching at λex=490 nm, λem=520 nm as a result of liposome destabilization and leakage of calcein into the surrounding medium. Addition of Triton X-100 to a final concentration of 1% following the 4 day incubation disrupts the liposome membranes achieving 100% calcein dequenching in order to calibrate the fluorescent scale: % leakage=((Iₙ−I₀)/(I₁₀₀−I₀))×100, where I₀ is the fluorescence at time 0, Iₙ is the fluorescence at time n, and I₁₀₀ is the totally dequenched calcein fluorescence following the addition of Triton.

For cellular uptake assays liposomes were prepared as previously described and included 1% (total moles) of rh-PE for monitoring their uptake in cells. For short-term liposome uptake assays in the presence or absence of serum, rh-PE-labeled liposomes were added to Huh7.5 cells grown to confluency in 6-well plates to a final phospholipid concentration of 50 μM in either serum-free complete DMEM, or complete DMEM supplemented with 10% FBS or 10% human serum (Sigma), and left to incubate for 24 h. Following the incubation, cells were washed twice in 1×PBS, counted, resuspended in 200 μl 1×PBS/0.5% Triton X-100, and transferred to a 96 well plate to read in a spectrofluorometer at λex=550 nm, λem=590 nm.

### 5.2. Results of Assays to Quantify Liposome Stability and Cellular Uptake in the Presence of 10% Serum

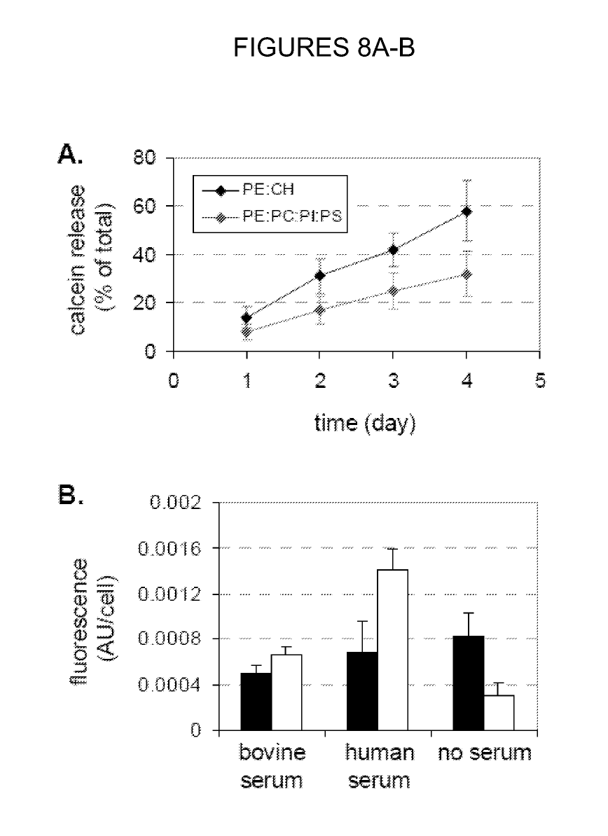

FIG. 8A demonstrates the rate of calcein release from within pH-sensitive DOPE:CH and ER-targeting DOPE:DOPC:PI:PS liposomes. Following a 4 day incubation, 58% (SD=12.6%) of calcein had been released from DOPE:PE liposomes, whereas only 32% (SD=9.2%) of calcein had leaked from DOPE:DOPC:PI:PS liposomes, suggesting greater stability in the presence of serum.

To monitor the effects of both FBS and human serum on the uptake of liposomes into Huh7.5 cells, DOPE:CH and DOPE:DOPC:PI:PS liposomes were prepared containing 1% rh-PE within the membrane, and incubated with Huh7.5 cells (final liposome concentration of 50 μM) for 24 h in the presence of serum-free media and media containing 10% FCS or 10% human serum. Liposome uptake in cells is expressed as the amount of fluorescence (in arbitrary units, AU) per cell following the 24 h incubation period. A significant decrease in DOPE:CH liposome uptake was observed in the presence of FBS compared to serum free media (5.0×10⁻⁴ AU/cell (SD=7.0×10⁻⁵ AU/cell) versus 8.2×10⁻⁴ AU/cell (SD=2×10⁻⁴ AU/cell), respectively, P=0.02, FIG. 8B). There was no significant difference in the presence of human serum. In contrast, DOPE:DOPC:PI:PS liposomes demonstrated a significant increase in uptake in the presence of FBS compared to serum-free media (6.6×10⁻⁴ AU/cell (SD=8.4×10⁻⁵ AU/cell) versus 3.1×10⁻⁴ AU/cell (SD=1.2×10⁻⁴ AU/cell), respectively, P=0.003, FIG. 8b). Furthermore, the presence of human serum significantly increased the efficiency of DOPE:DOPC:PI:PS liposome uptake in Huh7.5 cells compared to FBS (1.4×10⁻³ AU/cell (SD=1.8×10⁻⁴ AU/cell), P=0.001, FIG. 8B).

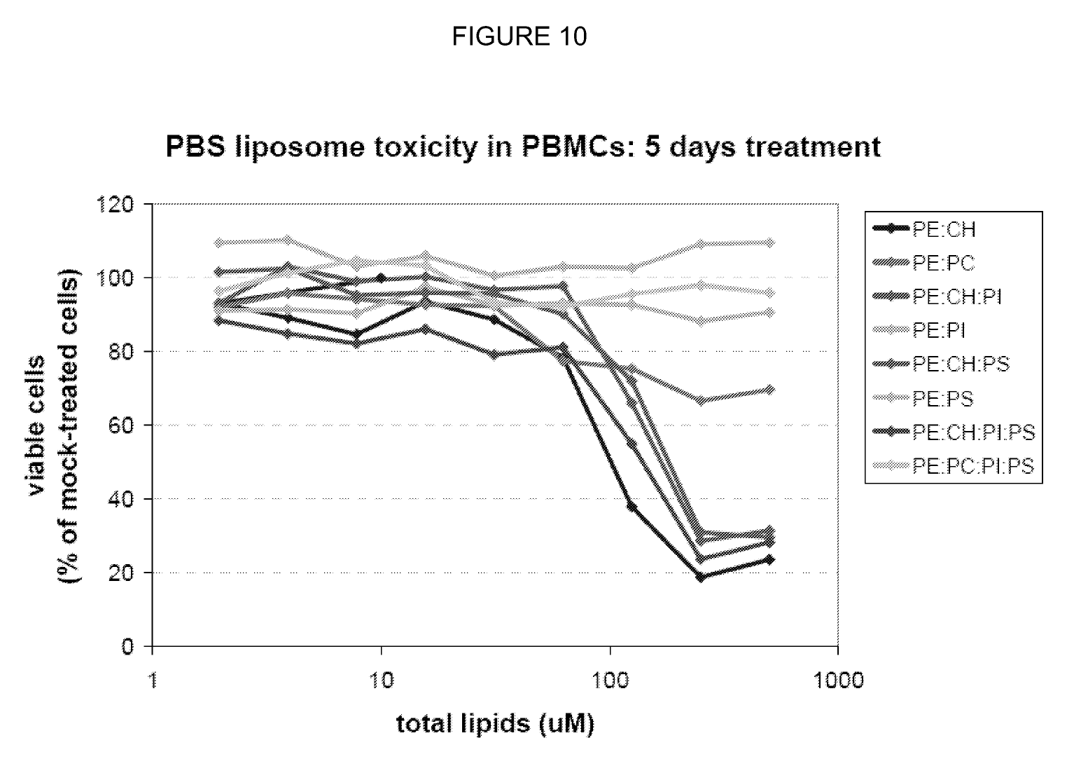

FIGS. 10A-B present results of experiments that demonstrate that ER-targeting liposomes have increased stability and cellular uptake in the presence of serum. (A) Self-quenching calcein-loaded liposomes (final lipid concentration of 50 μM) were incubated in complete DMEM+10% FBS, and left to incubate at 37° C. for 4 days. Every 24 h, a sample of the culture was used to measure calcein dequenching at λex=485 nm, λem=520 nm. Results are presented as the percentage of calcein released from liposomes in relation to the maximum fluorescence which is determined by the addition of Triton X-100 to disrupt the liposome membranes at the end of the incubation period. (B) Rh-labeled liposomes (50 μM lipid concentration) were incubated with Huh7.5 cells for 24 h in the presence of either 10% bovine serum (FBS), 10% human serum, or in serum-free media. Following the incubation time, cells were harvested, counted, and fluorescence was measured at λex=550 nm, λem=590 nm. Results are presented as the measured average fluorescence per cell for each sample. All data represent the mean and SD of triplicate samples from three independent experiments.

These studies using DOPE:DOPC:PI:PS liposomes can suggest that this phospholipid combination can demonstrate more favorable interactions with both cells and serum in comparison to DOPE:CH liposomes. In the presence of 10% FBS, DOPE:DOPC:PI:PS liposomes exhibit 45% less leakage of encapsulated cargo compared to DOPE:CH liposomes following a 4 day incubation. DOPE:DOPC:PI:PS liposomes also demonstrated increased uptake into Huh7.5 cells in the presence of FBS, which was further increased in the presence of human serum. In contrast, DOPE:CH liposome uptake appeared to be inhibited in the presence of FBS compared to serum-free medium. Although the present inventions are limited their theory of operation, these results can suggest that liposomes that target the ER, i.e. liposomes that contain PI and/or PS lipids, are endocytosed by different cellular receptors as those used by DOPE:CH liposomes, and that endocytosis via this mechanism can be enhanced by the presence of serum.

### 6. Cytotoxicity of ER Liposomes in Huh7.5 Cells and PBMCs

The purpose of these experiments was to determine the effect of liposomes on cell viability over one round of treatment (5 days) with both Huh7.5 cells and PBMCs.

### 6.1. Specific Methodology for Determination of Cytotoxicity in Huh7.5 Cells and PBMCs

Liposomes with the lipid composition PE:CH (3:2), PE:PC (3:2), PE:PI (3:2), PE:CH:PI (3:1:1), PE:PC:PI (1.5:1.5:2), PE:PS (3:2), PE:CH:PS (3:1:1), PE:PC:PS (1.5:1.5:2), PE:PI:PS (3:1:1), PE:CH:PI:PS (3:1:0.5:0.5) and PE:PC:PI:PS (1.5:1.5:1:1) were prepared as previously described. Huh7.5 cells and PBMCs were seeded in 96 well plates at a concentration of 5×10⁴ cells/well in 200 μl of complete DMEM and RPMI+IL2 medium, respectively, and incubated in the presence of liposomes encapsulating 1×PBS with final lipid concentrations in the range of 0-500 μM. After a 5 day incubation, cellular viability was determined by an MTS-based cell proliferation assay (CellTiter 96®, Promega, San Luis Obispo, U.S.A.) following the manufacturers' protocol.

### 6.2. Cytotoxicity in Huh7.5 Cells and PBMCs when Treated for 5 Days with PBS Liposomes

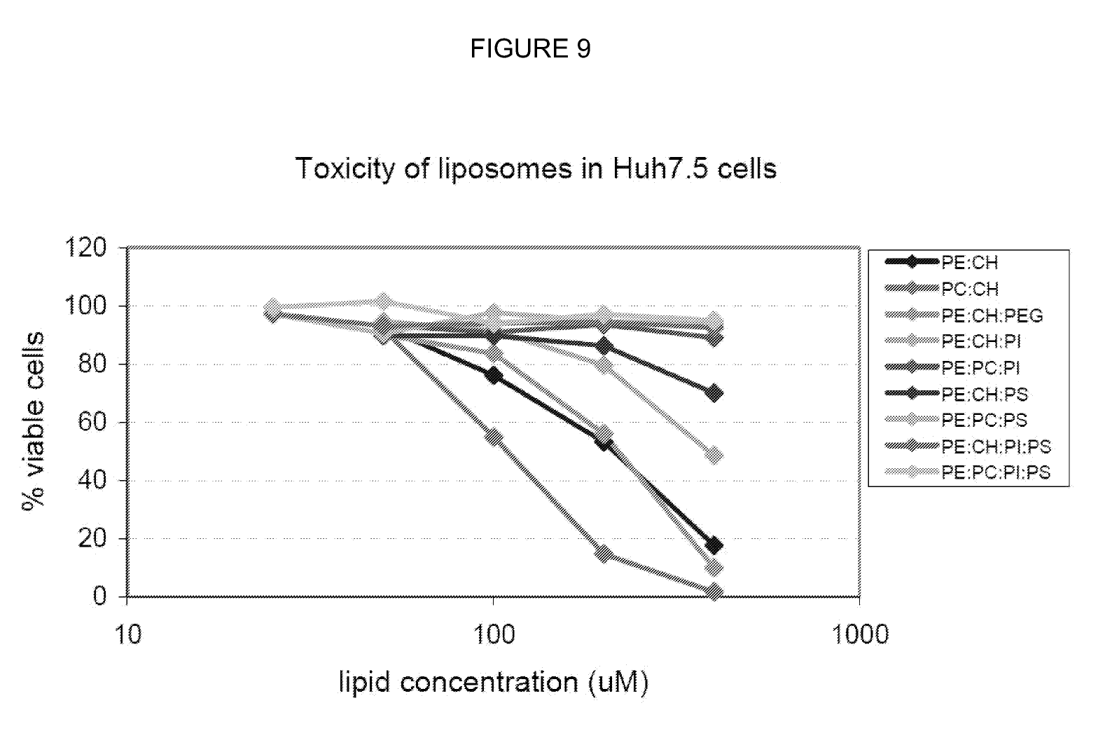

FIG. 9 shows viability of Huh7.5 cells following a 5 day incubation with different liposome formulations encapsulating 1×PBS. Final lipid concentrations in the medium ranged from 0 to 500 μM. Results represent the mean values of triplicate samples from three independent experiments.

FIG. 10 shows viability of PBMCs following a 5 day incubation with different liposome formulations encapsulating 1×PBS. Final lipid concentrations in the medium ranged from 0 to 500 μM. Results represent the mean values of triplicate samples from three independent experiments.

Results of FIGS. 9 and 10 can demonstrate that only liposomes containing the lipid CHEMS are cytotoxic in Huh7.5 cells and PBMCs when added to cells at concentrations greater than 60 μM. ER liposomes without this lipid show little cytotoxicity compared to pH-sensitive liposomes (PE:CH), if any, and are therefore preferable for in vivo uses.

### 7. Secretion of HIV-1 from Infected PBMCs Treated with ER-Liposomes

The purpose of these experiments was to monitor changes in the levels of HIV-1 secretion from HIV-1-infected PBMCs treated with different liposome compositions.

### 7.1. Specific Methodology for Single-Round HIV Secretion Assays

Liposomes with the lipid composition PE:CH (3:2), PE:PC (3:2), PE:PI (3:2), PE:CH:PI (3:1:1), PE:PS (3:2), PE:CH:PS (3:1:1), PE:CH:PI:PS (3:1:0.5:0.5) and PE:PC:PI:PS (1.5:1.5:1:1) were prepared as previously described. Changes in the secretion of HIV as a result of infection with virions secreted from drug-treated cells were assessed using stimulated PBMCs as indicator cells and determination of p24 antigen production as the end point. PBMCs from four normal (uninfected) donors were isolated using Histopaque density gradient centrifugation (Sigma-Aldrich, Gillingham, U.K.), pooled, and stimulated with phytohemagglutinin (PHA, 5 μg/ml) for 48 h followed by interleukin-2 (IL2, 40 U/ml) for 72 h in complete RPMI (RPMI plus 10% heat-inactivated FBS, 100 U/ml penicillin, 100 μg/ml streptomycin, and 2 mM L-glutamine). All experiments were performed in 96-well microtiter plates, and all incubations were at 37° C./5% CO₂, unless otherwise stated. To infect cells, 4×10⁵ PHA-activated PBMCs and 100 TCID₅₀ (tissue culture infectious dose 50%) of primary isolate stock were added to each well. Following an overnight incubation of 16 h, cells were washed three times with complete RPMI medium, and resuspended in complete RPMI/IL2 containing the appropriate free drug or liposome treatment (final lipid concentration of 50 μM). On day 5, supernatant containing HIV virions secreted from drug-treated cells is collected and p24 concentration is quantified for each by p24 capture ELISA.

### 7.2. Results from Single-Round HIV Secretion Assays

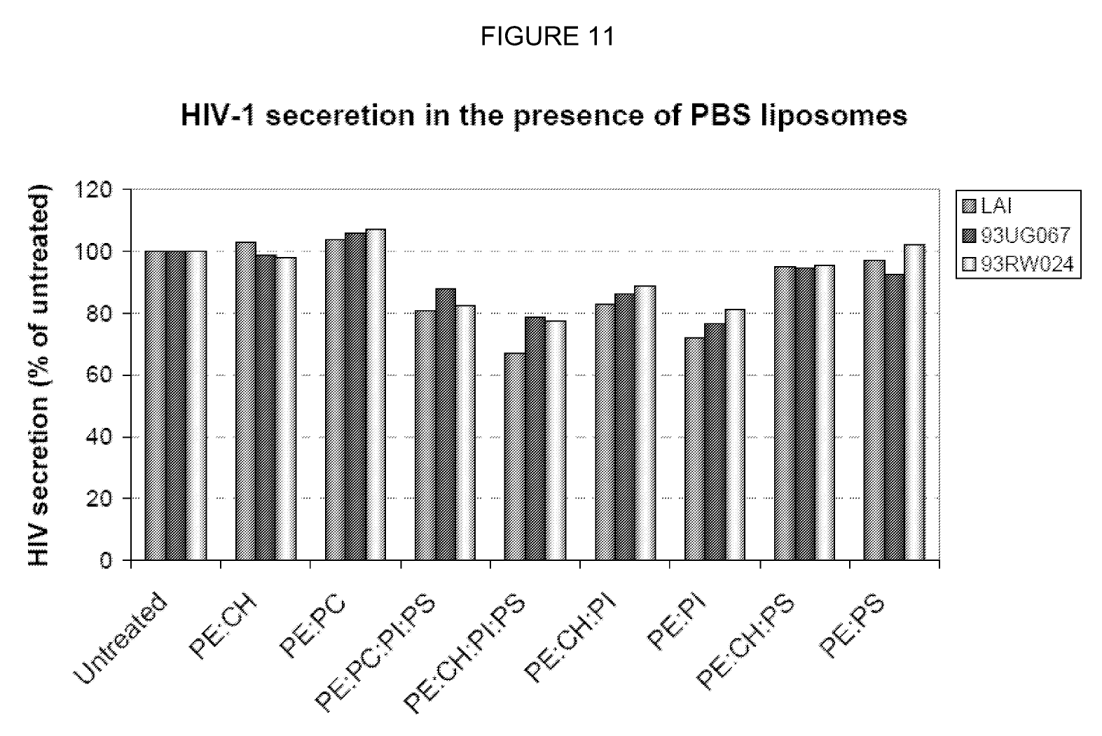

FIG. 11 demonstrates secretion of HIV from infected PBMCs during treatment with liposomes for 5 days. All liposomes are encapsulating a 1×PBS solution, and have been added to the cell culture media at a final lipid concentration of 50 μM. Viral secretion was calculated following the quantification of the HIV core protein, p24, within the supernatant of treated and untreated PBMCs by capture ELISA. Results are presented as the percent of HIV secretion in relation to the untreated control, and represent the average of triplicate samples from two independent experiments. The assay was conducted on three genetically diverse isolates of HIV-1, including LAI (clade B), 93UG067 (clade D) and 93RW024 (clade A).

Results in FIG. 11 can demonstrate that ER liposomes containing the lipid PI are capable of decreasing HIV secretion from PBMCs by approximately 20% compared to the untreated control. Non-ER targeting liposomes (PE:CH and PE:PC) and ER liposomes that do not contain a PI lipid have no effect on HIV secretion.

### 8. Infectivity of HIV-1 Secreted from Infected PBMCs Treated with ER Liposomes

The purpose of these experiments was to monitor changes in the infectivity of HIV-1 virions secreted from HIV-1-infected PBMCs treated with different liposome compositions.

### 8.1. Specific Methodology for Single-Round HIV Infectivity Assays

The infectivity of HIV virions secreted from PBMCs treated with liposomes was determined using supernatant containing HIV virions secreted from liposome-treated cells as described in the previous section. All supernatants were diluted to a final p24 concentration of 10 ng/ml in complete RPMI/IL2, and 100 μl was added to 4×10⁵ PHA-activated PBMCs, also in 100 μl of medium, for a final p24 concentration of 5 ng/ml, and left to incubate overnight. The following day cells were washed as described, resuspended in 200 μl of fresh RPMI/IL2, and left to incubate 4 days before supernatant was collected and assayed for p24 content by capture ELISA.

### 8.2. Results from Single-Round HIV Infectivity Assays

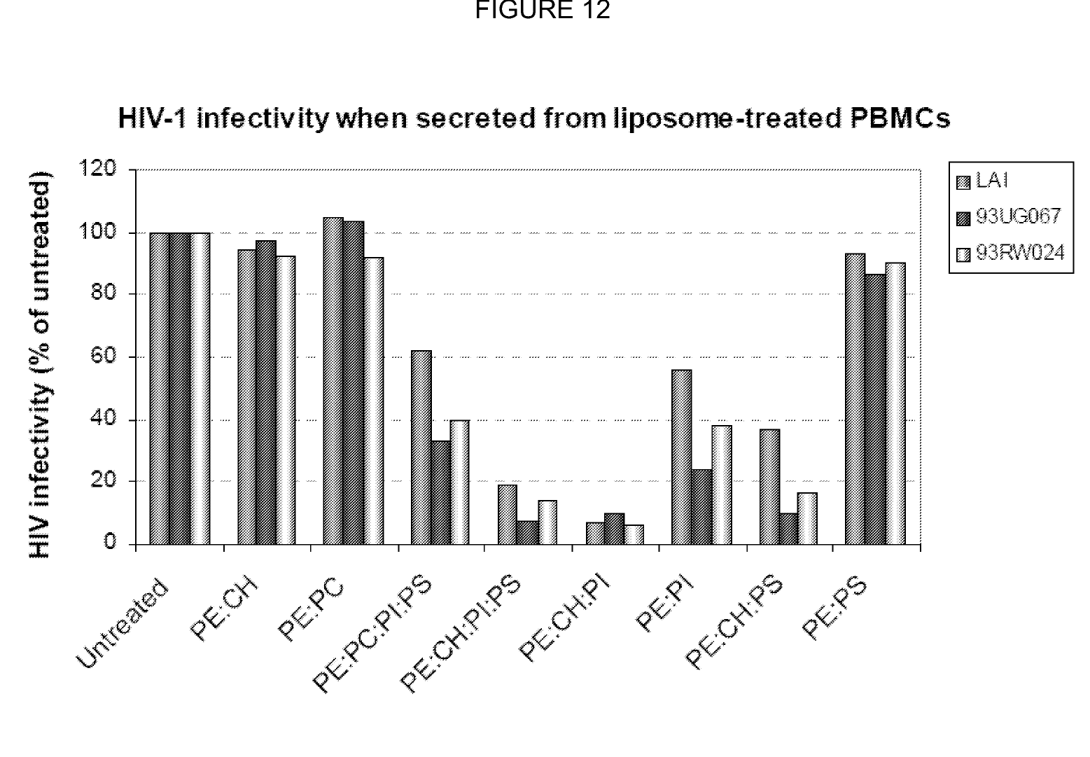

FIG. 12 shows the infectivity of HIV virions secreted from liposome-treated HIV-infected PBMCs. Secreted viral particles were used to infect naïve PBMCs, and the ability to infect cells was determined by measuring viral secretion once supernatant had been removed and cells were left untreated for 5 days. Results are presented as the percent of HIV infectivity in relation to the untreated control, and represent the average of triplicate samples from two independent experiments. The assay was conducted on three genetically diverse isolates of HIV-1, including LAI (clade B), 93UG067 (clade D) and 93RW024 (clade A).

Results in FIG. 12 can demonstrate that certain ER liposomes can be capable of reducing the infectivity of viral particles secreted from treated PBMCs. The greatest antiviral activity is seen with ER liposomes composed of the lipid CHEMS in combination with PI and/or PS, where infectivity of viral particles is less than 20% of the untreated virions. Non-ER liposomes (PE:CH and PE:PC) as well as the ER liposomes PE:PS had no effect on viral infectivity.

### 9. ER Liposomes Demonstrate More Efficient Intracellular Cargo Release Compared to pH-Sensitive Liposomes

In these experiments, rhodamine-labeled liposomes were prepared encapsulating a self-quenching concentration of calcein, a fluorescent molecule, and incubated in the presence of Huh7.5 cells. Delivery of encapsulated cargo inside cells was monitored by the increase in fluorescence as calcein is released into the intracellular space and becomes dequenched.

### 9.1 Specific Methodology for Measuring Intracellular Delivery of Liposomes in Huh7.5 Cells

For fluorometric assays, 5×10⁶ Huh7.5 cells were seeded into 25 cm² flasks in complete DMEM/10% FBS overnight. The following day, calcein-loaded liposomes containing 1% rh-PE were added to the medium (final phospholipid concentration of 50 μM) and left to incubate 30 min at 37° C. or 4° C. Following incubation, cells were washed twice in 1×PBS, detached with trypsin/EDTA (Invitrogen), washed twice more, and resuspended in 600 μl PBS. Three aliquots of 200 μl where used to take fluorometric measurements and were averaged. Calcein dequenching was measured at λex=485 nm and λem=520 nm, and rhodamine fluorescence was measured at λex=550 nm and λem=590 nm. The initial calcein to rhodamine fluorescence ratio of liposomes bound to cells in the absence of endocytosis was obtained by incubating the liposomes with cells at 4° C. and is used to adjust values at 37° C.

### 9.2. Intracellular Release of Encapsulated Calcein from Liposomes in Huh7.5 Cells

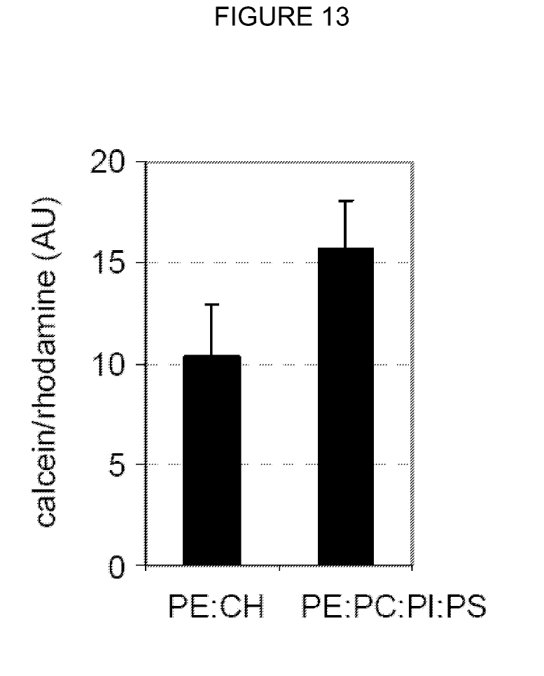

Mean rh-DOPE fluorescence in Huh7.5 cells following a 45 min incubation with liposomes reflects the uptake of liposomes, and the mean calcein fluorescence indicates intracellular dequenching, and therefore release of fluorescent dye. The calculated ratio of calcein to rhodamine fluorescence is taken as a measure of the amount of aqueous marker released intracellularly per cell-associated liposome. The calcein/rhodamine ratio for DOPE:CH liposomes was calculated to be 10.3 (SD=2.6), whereas the ratio for DOPE:DOPC:PI:PS liposomes was 15.7 (SD=2.4), an increase of 152% (P=0.02, FIG. 13).

FIG. 13 presents results of experiments for self-quenching calcein-loaded, rh-PE-labeled, liposomes (final lipid concentration of 50 μM) incubated with Huh7.5 cells in complete DMEM/10% FBS for 45 min. Intracellular dequenching of calcein from liposomes following the incubation was measured at λex=490 nm, λem=520 nm, and the total liposome uptake during the same incubation period was determined by fluorescent measurements at λex=550 nm, λem=590 nm. The assay was conducted both at 37° C. and 4° C., and to correct for liposome binding without endocytosis, all 4° C. values were subtracted from the 37° C. values. The ability of liposomes to deliver encapsulated calcein inside Huh7.5 cells was measured by calculating the ratio of calcein dequenching and rh-PE fluorescence in treated cells following the incubation. Data represent the mean and SD of triplicate samples from three independent experiments.

Results presented in FIG. 13 can suggest that liposomes composed of PE in combination with PI and/or PS have increased levels of intracellular calcein release per liposome compared to PE:CH liposomes, a liposome composition specifically designed for efficient intracellular delivery of encapsulated compounds. In these assays, PE:PC:PI:PS liposomes demonstrate 1.5 times greater calcein release compared to PE:CH liposomes.

### 10. Secretion of HIV-1 from PBMCs Treated with Liposomes Encapsulating 1 Mm NB-DNJ

The purpose of these experiments was to determine the ability of liposomes to deliver encapsulated iminosugars (i.e. NB-DNJ) to HIV-infected PBMCs. Liposomes containing the lipids PI and PS were compared to pH-sensitive liposomes (PE:CH) and pH-insensitive liposomes (PE:PC).

### 10.1. Specific Methodology for Single-Round HIV Secretion Assays

Liposomes with the lipid composition PE:CH (3:2), PE:PC (3:2), PE:PI (3:2), PE:CH:PI (3:1:1), PE:PS (3:2), PE:CH:PS (3:1:1), PE:CH:PI:PS (3:1:0.5:0.5) and PE:PC:PI:PS (1.5:1.5:1:1) were prepared as previously described, except all liposomes encapsulated 1 mM NB-DNJ in 1×PBS. HIV secretion assays were carried out as previously described. Liposomes were purified from unencapsulated NB-DNJ by size-exclusion chromatography. Results with liposomes are compared to those with NB-DNJ added to a final concentration of 1 mM in the cell culture media.

### 10.2 Results from Single-Round HIV Secretion Assays

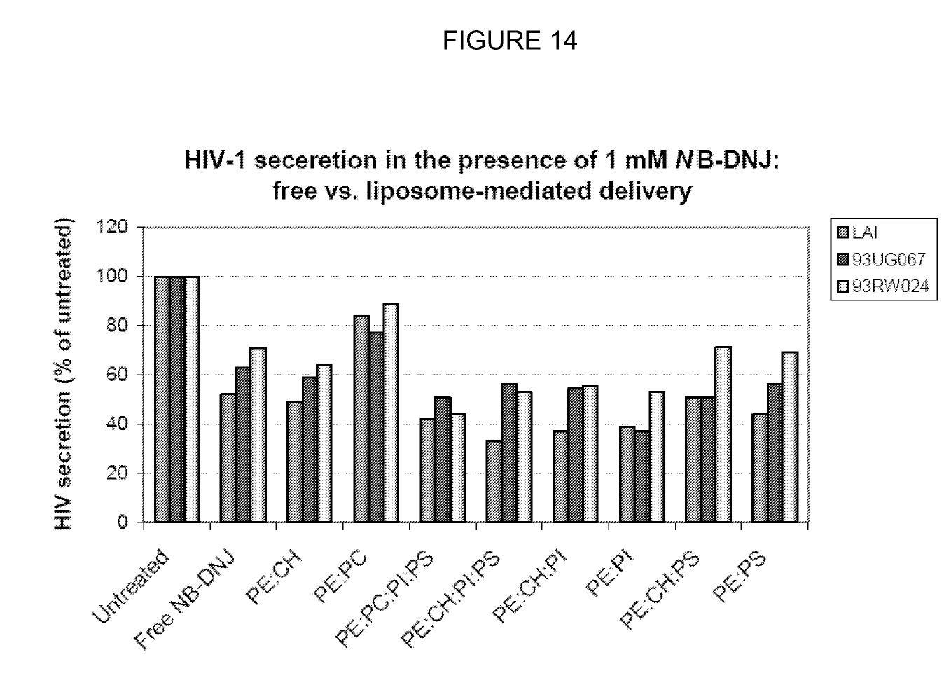

FIG. 14 shows secretion of HIV from infected PBMCs during a 5 day treatment with 1 mM NB-DNJ: free vs. liposome-mediated delivery. Liposomes are encapsulating 1 mM NB-DNJ, and have been added to the cell culture media at a final lipid concentration of 50 μM. Viral secretion was calculated as previously described. Results are presented as the percent of HIV secretion in relation to the untreated control, and represent the average of triplicate samples from two independent experiments. The assay was conducted on three genetically diverse isolates of HIV-1, including LAI (clade B), 93UG067 (clade D) and 93RW024 (clade A).

Results in FIG. 14 can demonstrate that liposomes containing the lipids PI or PS can be capable of delivering the antiviral NB-DNJ to HIV-infected PBMCs to achieve similar, if not better, antiviral activity compared to PE:CH liposomes as determined by the decrease in HIV secretion.

### 11. Infectivity of HIV-1 Secreted from Infected PBMCs Treated with Liposomes Encapsulating 1 mM NB-DNJ

The purpose of these experiments was to determine the ability of liposomes to deliver encapsulated iminosugars (i.e. NB-DNJ) to HIV-infected PBMCs. Liposomes containing the lipids PI and PS were compared to pH-sensitive liposomes (PE:CH) and pH-insensitive liposomes (PE:PC).

### 11.1. Specific Methodology for Single-Round HIV Infectivity Assays

The infectivity of HIV virions secreted from PBMCs treated with liposomes was determined as described previously, except all liposomes encapsulated 1 mM NB-DNJ in 1×PBS. Results with virions secreted from liposome-treated cells are compared to those from free NB-DNJ-treated cells and untreated cells.

### 11.2. Results from Single-Round HIV Infectivity Assays

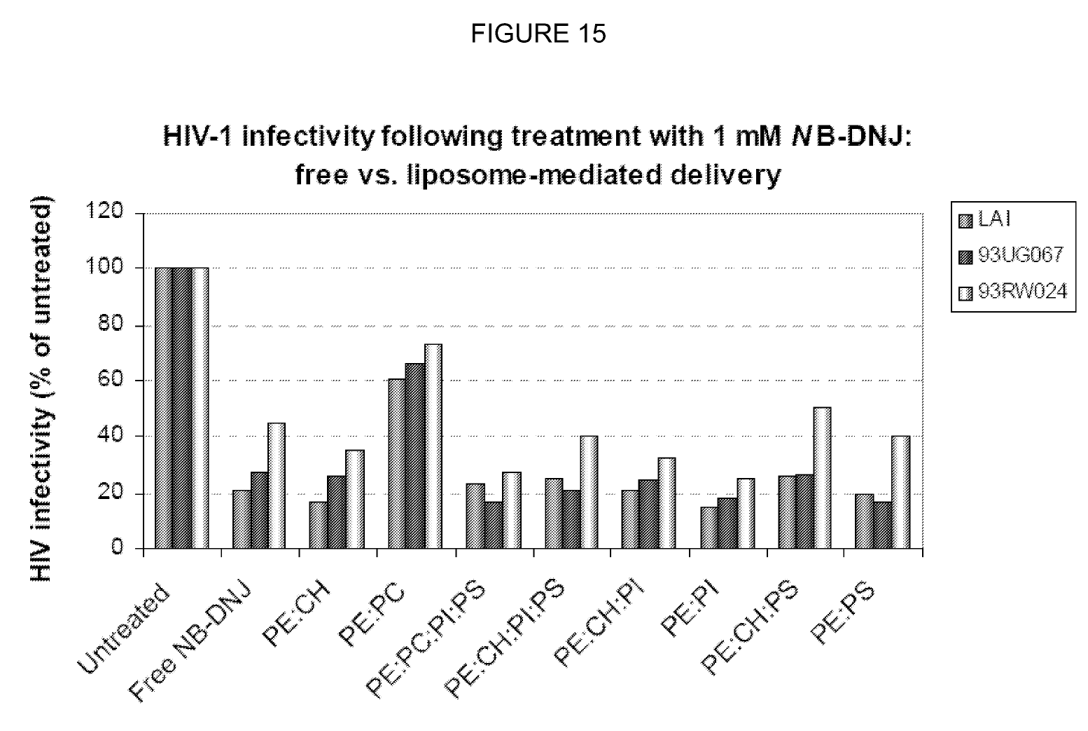

FIG. 15 shows the infectivity of HIV virions secreted from NB-DNJ-liposome or free NB-DNJ-treated HIV-infected PBMCs. Secreted viral particles were used to infect naïve PBMCs, and the ability to infect cells was determined as previously described. Results are presented as the percent of HIV infectivity in relation to the untreated control, and represent the average of triplicate samples from two independent experiments. The assay was conducted on three genetically diverse isolates of HIV-1, including LAI (clade B), 93UG067 (clade D) and 93RW024 (clade A).

Results in FIG. 15 can demonstrate that treatment of HIV-infected PBMCs with ER liposomes encapsulating 1 mM NB-DNJ decrease the secretion and infectivity of HIV compared to the untreated control. Comparing results between pH-sensitive liposomes, which are liposomes that do not contain PI and PS lipids, and liposomes containing the lipids PI and PS reveals no significant differences in antiviral activity when encapsulating 1 mM NB-DNJ.

Antiviral activity can be further enhanced by chemically linking a gp120/gp41 targeting molecule, such as a soluble form of CD4, to the outer surface of drug-encapsulating liposomes. The targeting molecule should lead to the increased uptake of drug-loaded liposomes into HIV-infected cells via receptor-mediated endocytosis, in addition to neutralizing free viral particles preventing infection.

### 12. Cytotoxicity of EE-Liposomes Encapsulating 1 Mm NB-DNJ in PBMCs

The purpose of these experiments was to determine the effect of liposomes encapsulating 1 mM NB-DNJ on cell viability over one round of treatment (5 days) with PBMCs.

### 12.1. Specific Methodology for Determination of Cytotoxicity in PBMCs

Liposomes with the lipid composition PE:CH (3:2), PE:PC (3:2), PE:PI (3:2), PE:CH:PI (3:1:1), PE:PS (3:2), PE:CH:PS (3:1:1), PE:CH:PI:PS (3:1:0.5:0.5) and PE:PC:PI:PS (1.5:1.5:1:1) were prepared as previously described, except all liposomes encapsulated 1 mM NB-DNJ in 1×PBS. Cell viability following a 5 day incubation with liposomes encapsulating 1 mM NB-DNJ was determined as previously described.

### 12.2. PBMC Viability Following Treatment with Liposomes Encapsulating 1 Mm NB-DNJ

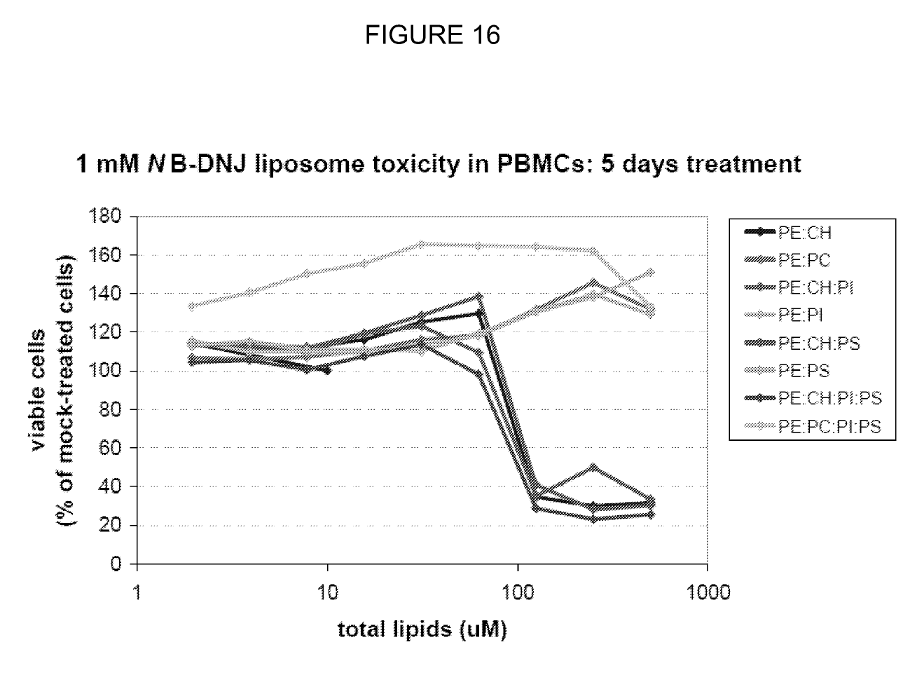

FIG. 16 shows viability of PBMCs following a 5 day incubation with different liposome formulations encapsulating 1 mM NB-DNJ. Final lipid concentrations in the medium ranged from 0 to 500 μM. Results represent the mean values of triplicate samples from three independent experiments.

Results in FIG. 16 demonstrate that the encapsulation of 1 mM NB-DNJ inside liposomes does not have additional cytotoxic activity. Surprisingly, encapsulation of NB-DNJ inside certain liposomes appears to increase cell proliferation to 160% compared to the mock-treated control.

### 13. Secretion of HCV from Huh7.5 Cells Treated with Er Liposomes

The purpose of these experiments was to monitor changes in the levels of HCV-1 secretion from HCV-infected Huh7.5 cells treated with different liposome compositions.

### 13.1. Method for Single Round HCV Secretion Assay

Assays were performed on cells 8 days post infection (acute) and 50 days post infection (chronic). HCV-infected Huh7.5 cells were grown to 75% confluency in 6 well plates, before media was replaced with complete DMEM+50 μM liposomes in a total volume of 2 ml per well and left to incubate for 72 h at 37° C./5% CO₂. All assays were performed with samples in triplicate. Virus secretion analysis was performed by quantitative PCR on viral RNA extracted from 500 μl of supernatant using the QIAGEN QIAamp Viral RNA Purification Kit, following the manufacturers' protocol. Quantification of secreted viral RNA was done by first converting isolated RNA to cDNA using a reverse transcriptase reaction followed by real-time PCR using a SyBr Green mix and primers directed against the HCV cDNA.

### 13.2. Results from Single Round Secretion Assays

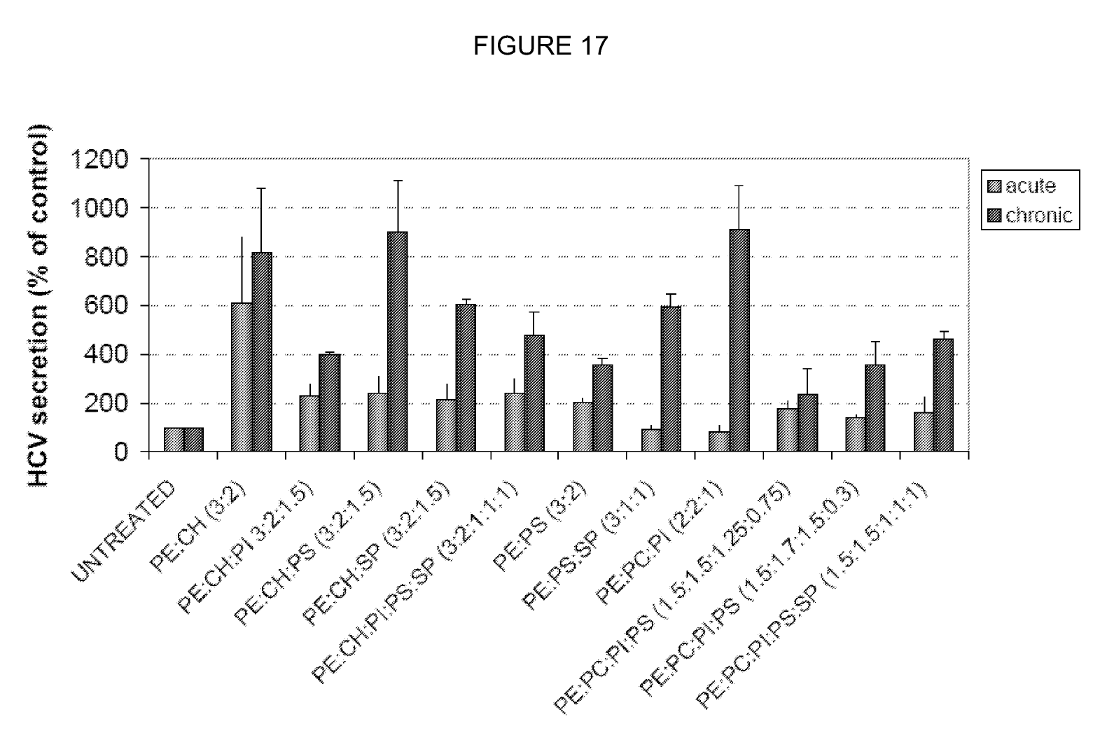

FIG. 17 shows secretion of HCV from infected Huh7.5 cells, both acutely and chronically-infected, following treatment with liposomes for 5 days. All liposomes are encapsulating a 1×PBS solution, and have been added to the cell culture media at a final lipid concentration of 50 μM. HCV secretion was calculated following the quantification of RNA within the supernatant of treated and untreated Huh7.5 cells by quantitative PCR. Results are presented as the percent of HCV RNA secretion in relation to the untreated control, and represent the average of triplicate samples.

### 14. Infectivity of HCV Secreted from Huh7.5 Cells Treated with ER Liposomes

The purpose of these experiments was to monitor changes in the infectivity of HCV virions secreted from HCV-infected Huh7.5 cells treated with different liposome compositions.

### 14.1. Method for Single-Round HCV Infectivity Assay

The infectivity of HCV virions secreted from Huh7.5 cells treated with liposomes was determined using supernatant containing HCV virions secreted from liposome-treated cells as described in the previous section. Naïve Huh7.5 cells were grown to 75% confluency in 48-well plates before medium was replaced with 200 μl of supernatant containing HCV secreted from liposome-treated cells. The supernatant was left to infect naïve Huh7.5 cells for 1 h before cells were washed twice with 1×PBS and then incubated in 500 μl complete DMEM for 2 days at 37° C./5% CO₂. After the 2 day incubation, cells were washed twice with 1×PBS, fixed in methanol/acetone (1:1, vol/vol) for 10 min, and washed twice in 1×PBS/0.1% Tween-20. Cells were then incubated for 1 h in 1×PBS/0.1% Tween-20 containing 4 μg/ml anti-HCV core antibody, washed twice in 1×PBS/0.1% Tween-20, incubated 1 h in 1×PBS/0.1% Tween-20 containing 4 μg/ml FITC-labeled secondary antibody, and washed twice more, and stained with DAPI. Fluorescent images were taken using a Nikon Eclipse TE2000-U microscope as previously described. The percentage of infected cells is calculated by counting the total number of cells infected with HCV (detected by the anti-HCV antibody) divided by the total number of cells in the assay (detected by DAPI staining).

### 14.2. Results from Single-Round HCV Infectivity Assays

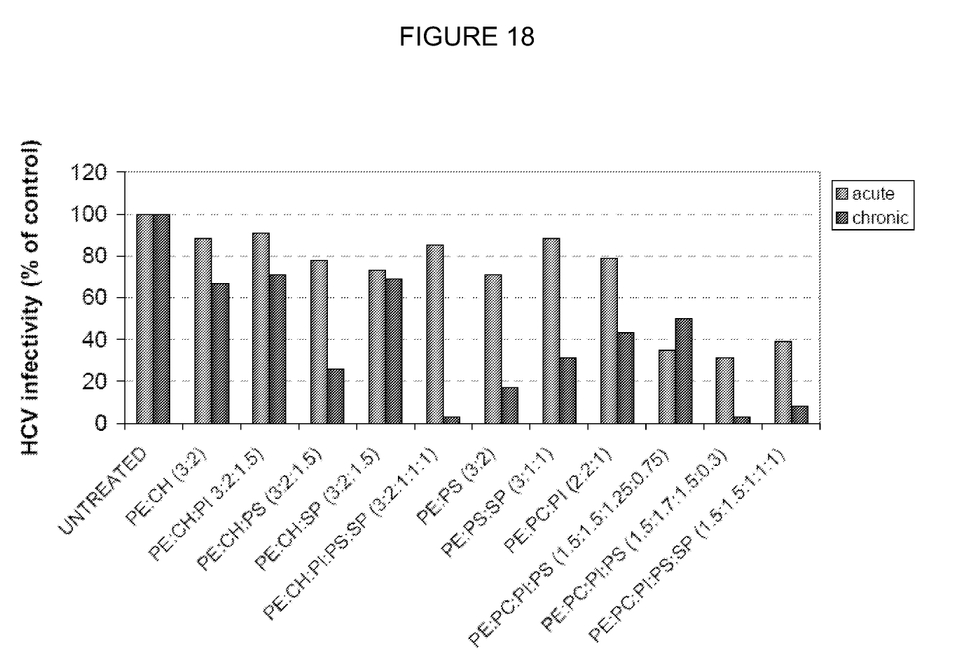

FIG. 18 shows the infectivity of HCV virions secreted from liposome-treated HCV-infected Huh7.5 cells, both acutely and chronically-infected. Secreted viral particles were used to infect naïve Huh7.5 cells, and the ability to infect cells was determined by measuring the presence of HCV core protein in naïve cells once supernatant had been removed and cells were left untreated for 2 days. Results are presented as the percent of HCV infectivity in relation to the untreated control, and represent the average of triplicate samples.

Results from HCV-infected Huh7.5 cells treated with a selection of ER liposomes and pH-sensitive liposomes (PE:CH) suggests that all liposomes increase the secretion of viral particles, however, the infectivity of the secreted particles are significantly reduced compared to untreated particles.

### 15. ER Liposomes Decrease the Formation of LDs in Huh7.5 Cells

Huh7.5 cells were incubated overnight in the presence of ER liposomes to monitor their effects on cellular LDs. LDs were visualized in liposome-treated cells by confocal microscopy.

### 15.1. Method for Visualizing LDs within Huh7.5 Cells

ER liposomes PE:PC:PI:PS (1.5:1.7:1.5:0.3) were prepared as previously described. Huh7.5 cells were allowed to adhere overnight onto number 1.5 glass cover slides before media was exchanged and replaced with fresh media containing liposomes added to a final lipid concentration of 50 μM. After a 16 h incubation at 37° C./5% CO₂, media containing liposomes were removed and cells were washed with 1×PBS, fixed in 4% paraformaldehyde diluted in 1×PBS for 15 min, and washed twice in 1×PBS. Cells were then incubated with 1×PBS containing 20 μg/ml of BODIPY493/503 for 10 min and washed twice in 1×PBS. BODIPY 493/503 is appropriate for detailed analyses of microenvironments around the LD. Cells were stained with DAPI prior to mounting onto microscope slides. Confocal images were taken using a Carl Zeiss LSM microscope, and image analysis was done using the LSM software v5.10.

### 15.2. Results from Visualizing LDs Inside Huh7.5 Cells Following a 16 h Treatment with ER Liposomes

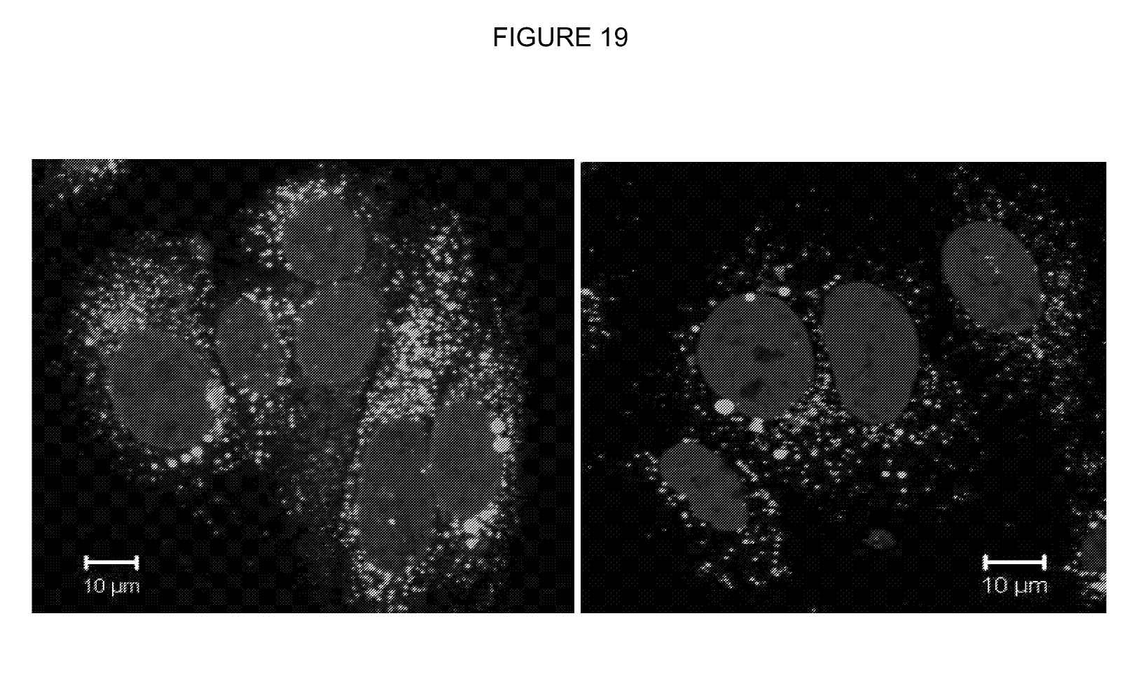

FIG. 19 shows results of experiments for untreated Huh7.5 cells (left panel) and PE:PC:PI:PS liposome-treated Huh7.5 cells (right panel) probed with BODIPY 493/503 (green) to visualize LDs following a 16 h incubation. PE:PC:PI:PS liposomes were added to the cell culture media to a final lipid cincentration of 50 μM. DAPI (blue) is used as a nuclear stain and to normalize image intensity.

Results suggest that treatment of Huh7.5 cells with PE:PI:PS:PC liposomes decrease the formation of LDs.

### 16. ER Liposomes Co-Localize with LDs in Huh7.5 Cells

Since PE:PC:PI:PS liposomes were shown to interfere with LD formation in Huh7.5 cells, the following experiment was performed to determine if these liposomes directly interact with cellular LDs. Rh-PE labeled liposomes were incubated with Huh7.5 cells for 2 h before Rh-PE lipids and cellular LDs were visualized by confocal microscopy to determine co-localization.

### 16.1. Method for Visualizing the Intracellular Co-Localization of LDs and Liposomes

ER liposomes PE:PC:PI:PS (1.5:1.7:1.5:0.3) were prepared as previously described and included 1% (total moles) of Rh-PE for visualization. Huh7.5 cells were allowed to adhere overnight onto number 1.5 glass cover slides before media was exchanged and replaced with fresh media containing Rh-PE labeled liposomes added to a final lipid concentration of 50 μM. After a 2 h incubation at 37° C./5% CO₂, media containing liposomes were removed and cells were fixed and stained with BODIPY 493/503 as previously described. Cells were stained with DAPI prior to mounting onto microscope slides. Confocal images were taken as previously described

### 16.2. Co-Localization of Huh7.5 LDs with Liposomes Following a 2 h Incubation

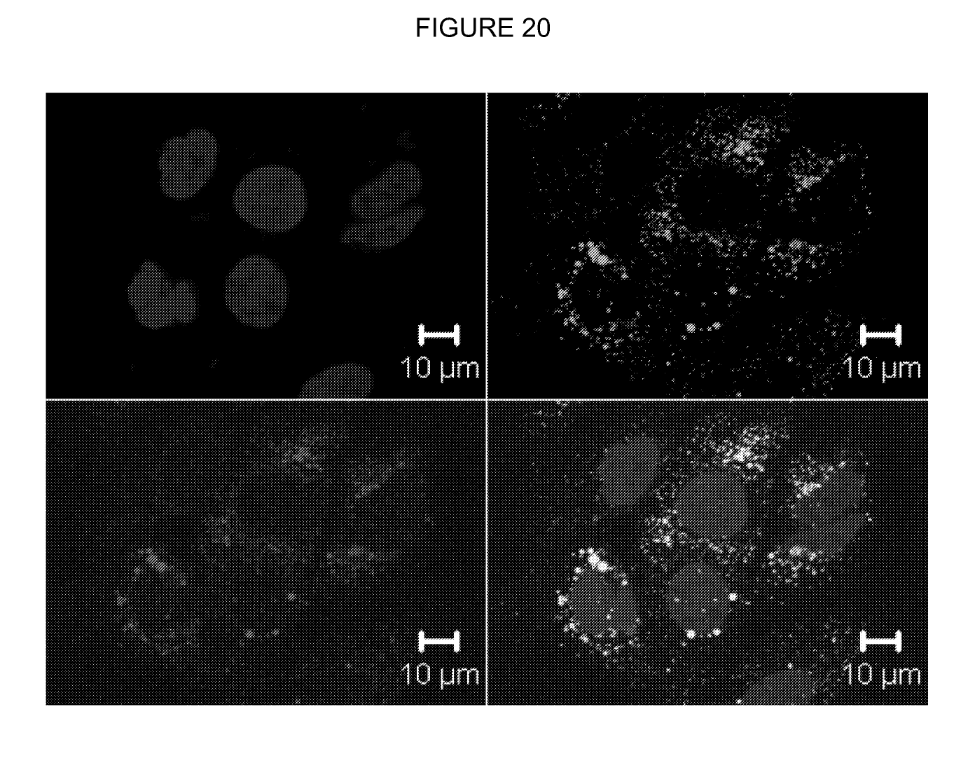

FIG. 20 shows results of experiments for Huh7.5 cells treated with PE:PC:PI:PS liposomes (red) for 2 h and probed with a LD stain (green). PE:PC:PI:PS liposomes were added to the cell culture media to a final lipid cincentration of 50 μM. DAPI (blue) is used as a nuclear stain. Bottom-right panel is the merged image. Yellow colour identifies areas of co-localization within the cell.

Results suggest that PE:PI:PS:PC liposomes can interact with LDs in Huh7.5 cells following only 2 hours of treatment.

### 17. Treatment of HCV-Infected Huh7.5 Cells with Er Liposomes Inhibits the Association of HCV Core Protein with LDs

Interfering with the interaction between HCV core protein and cellular LDs can lead to the secretion of primarily non-infectious viral particles from HCV-infected cells. The purpose of these experiments was to determine if liposome treatment reduces the co-localization of the HCV core protein and LDs in Huh7.5 cells.

### 17.1. Method for Visualizing the Intracellular Co-Localization of LDs and HCV Core Protein

ER liposomes PE:PC:PI:PS (1.5:1.7:1.5:0.3) were prepared as previously described. Huh7.5 cells, 8 days post-infection with HCV genotype JFH1, were allowed to adhere overnight onto number 1.5 glass cover slides before media was exchanged and replaced with fresh media containing liposomes added to a final lipid concentration of 50 μM. After a 16 h incubation at 37° C./5% CO₂, media containing liposomes were removed and cells were washed twice with 1×PBS, fixed in methanol/acetone (1:1, vol/vol) for 10 min, and washed twice in 1×PBS/0.1% Tween-20. Cells were then incubated for 1 h in 1×PBS/0.1% Tween-20 containing 3 μg/ml anti-HCV core antibody, washed twice in 1×PBS/0.1% Tween-20, incubated 1 h in 1×PBS/0.1% Tween-20 containing 4 μg/ml AlexaFluor 550-labeled secondary antibody, and washed twice more. Cells were then incubated with 1×PBS containing 20 μg/ml of BODIPY493/503 for 10 min and washed twice in 1×PBS prior to DAPI staining and mounting as previously described. Confocal images were taken as previously described.

### 17.2. Co-Localization of Huh7.5 LDs with the HCV Core Protein Following a 16 h Treatment with ER Liposomes

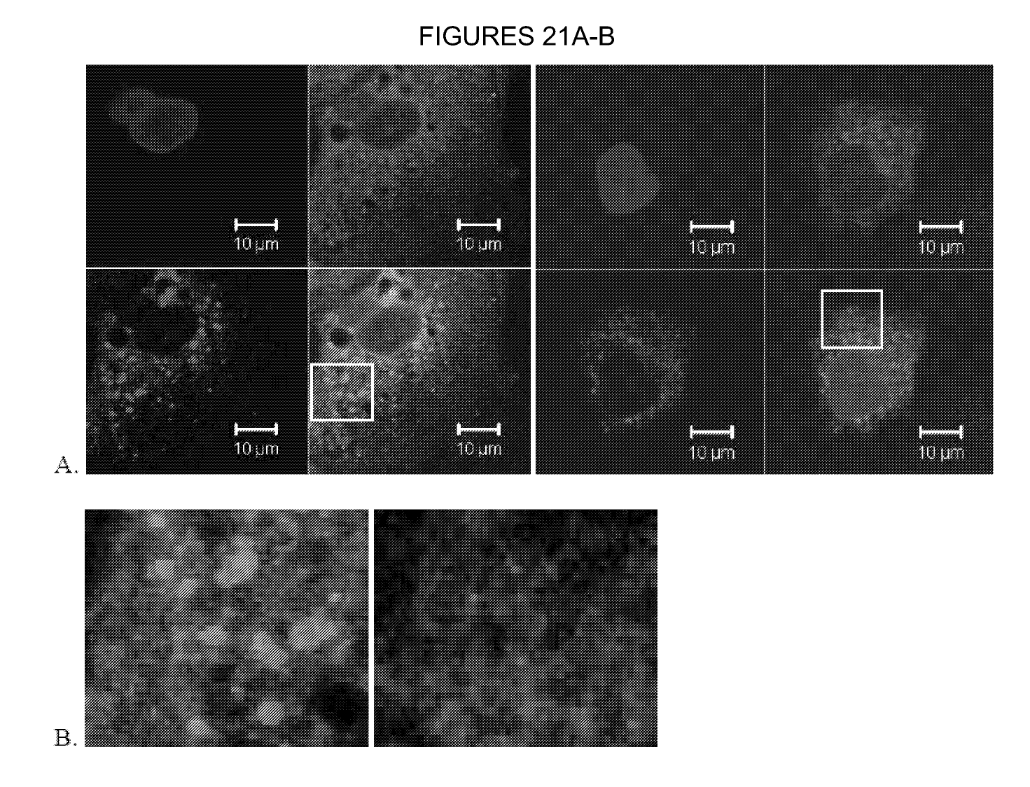

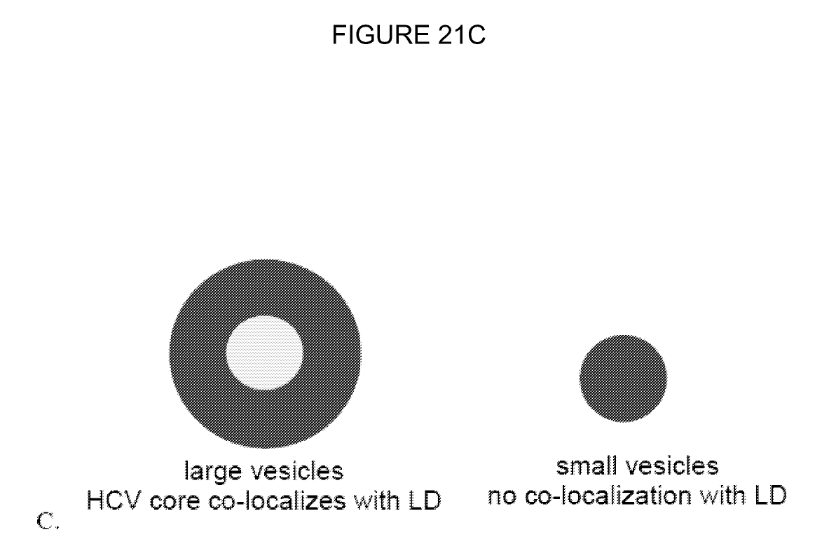

FIG. 21A shows results of experiments for untreated Huh7.5 cells (left panel) and PE:PC:PI:PS liposome-treated Huh7.5 cells (right panel) were incubated for 16 h and probed with an anti-HCV core antibody (red) and an LD stain (green). PE:PC:PI:PS liposomes were added to the cell culture media to a final lipid cincentration of 50 μM. DAPI (blue) is used as a nuclear stain. Bottom-right panel is the merged image. Yellow colour identifies areas of co-localization within the cell. FIG. 21B presents close-up of merged images (white boxes) for both untreated (left) and PE:PC:PI:PS liposome-treated (right) cells. FIG. 21C is a schematic representation of the HCV core protein/LD interaction in the presence (right) and absence (left) of PE:PC:PI:PS liposomes.

The presence of large LD/HCV core vesicles may be necessary for the production of infectious viral particles. These results demonstrate that treatment of HCV-infected Huh7.5 cells with PE:PC:PI:PS liposomes can reduce the association of HCV core with cellular LDs, which most likely can explain the decrease in infectivity of HCV particles secreted from ER liposome-treated cells.

### 18. Decreasing HCV Secretion and Infectivity by Delivering Polyunsaturated Lipids Via ER Liposomes to HCV-Infected Huh7.5 Cells

To enhance the antiviral activity of ER liposomes against HCV, the PE and PC lipids (currently 18:1 monounsaturated in all experiments) can be replaced with polyunsaturated PE and PC (either 22:6 and/or 20:4).

FIGS. 22A-D shows chemical structures of polyunsaturated lipids to be incorporated into polyunsaturated ER liposomes. A. 22:6 PE B. 20:4 PE. C. 22:6 PC. D. 20:4 PC. To investigate the potential role of ER liposomes as HCV antivirals, JC-1-infected Huh7.5 cells were treated with various liposome compositions to monitor their effect on HCVcc secretion and infectivity. In addition to 22:6 ER liposomes (22:6 PE:22:6 PC:PI:PS, 1.5:1.5:1:1) and 22:6 PEG-ER liposomes (22:6 polyunsaturated ER liposomes containing 3% PEG-PE lipids), 20:4 ER liposomes (20:4 PE:20:4 PC:PI:PS, 1.5:1.5:1:1) and 18:1 ER liposomes (18:1 PE:18:1 PC:PI:PS, 1.5:1.5:1:1) were included to monitor the effect of different liposome lipid saturations on HCV replication.

### 18.1. Methodology for Monitoring HCVcc Secretion and Infectivity Following Liposome Treatment

Methods are identical to those described above in sections 13 & 14 for an acute JC-1 HCVcc infection.

### 18.2. HCVcc Secretion During a 4 Day Treatment with Liposomes

As demonstrated in FIG. 23A, both 18:1 and 20:4 lipids led to an increase in HCVcc secretion compared to untreated control samples (218%, SD=34.4%, and 159%, SD=21.6%, respectively). Only 22:6 ER liposomes were shown to significantly decrease HCV secretion by 27% (SD=11.3%) at a concentration of 50 μM; a similar decrease was observed with 50 μM 22:6 PEG-ER liposome-treatment (23%, SD=6.6%). To measure the infectivity of secreted viral particles, supernatant from liposome-treated HCVcc was used to infect naïve Huh7.5 cells, and the number of infected cells was quantified 48 h post-infection. FIG. 23B shows a significant decrease in HCV infectivity with all liposome treatments, even with the 18:1 and 20:4 ER liposome treatments which caused increased viral secretion. Treatment with 50 μM 22:6 ER liposomes decreased HCV infectivity by 91% (SD=2.2%). Even the lowest concentration of 22:6 ER liposomes tested, 1 μM, decreased infectivity by 52% (SD=5.3%), suggesting 22:6 polyunsaturated (pu) ER liposomes are potent inhibitors of viral infectivity.

FIG. 23A shows JC-1 HCVcc secretion from infected Huh7.5 cells (MOI=0.5) during a 4 day incubation in the presence of various ER liposome formulations was quantified from 500 μl of cellular supernatant. Secretion is measured by the quantification of JC-1 HCVcc RNA within the supernatant by quantitative PCR. FIG. 23B shows infectivity of secreted JC-1 HCVcc from liposome-treated, JC-1-infected Huh7.5 cells. Infectivity of the secreted HCVcc was determined by infection of naïve Huh7.5 cells for 1 h, followed by a 48 h incubation at which point cells were fixed and stained with an anti-HCV core antibody to quantify the number of infected cells, and DAPI to visualize all cells.

The data in FIGS. 23A-B can suggest that ER liposomes containing the lipids 22:6 can significantly decrease the infectivity of secreted HCV virions similar to the previously described ER liposomes (18:1 lipids). ER liposomes composed 22:6 polyunsaturated lipids are currently the favorite for development into an anti-HCV therapy.

Although the foregoing refers to particular preferred embodiments, it will be understood that the present invention is not so limited. It will occur to those of ordinary skill in the art that various modifications may be made to the disclosed embodiments and that such modifications are intended to be within the scope of the present invention.

All of the publications, patent applications and patents cited in this specification are incorporated herein by reference in their entirety.

What is claimed is:

1. A method of drug delivery, comprising
administering to a host in need thereof a composition comprising a lipid particle comprising at least one PS lipid.

2. The method of claim 1, wherein the lipid particle is a liposome.

3. The method of claim 1, wherein the lipid particle further comprises at least one of PE, CHEMS, PI, PC or SP lipids.

4. The method of claim 3, wherein the lipid particle comprises PE lipids and a molar ratio between the PE lipids and the PS lipids ranges from 0.5:1 to 20:1.

5. The method of claim 4, wherein the molar ratio between the PE lipids and the PS lipids in the lipid particle ranges from 1:1 to 10:1.

6. The method of claim 5, wherein the PE lipids comprise DOPE lipids and PEG-PE lipids.

7. The method of claim 3, wherein the lipid particle comprises PE, PI and PC lipids.

8. The method of claim 1 applied for treating or preventing a disease or condition caused by or associated with a virus.

9. The method of claim 8, wherein the disease or condition is a viral infection.

10. The method of claim 8, wherein said administering results in incorporating one or more lipids of the lipid particle into an endoplasmic reticulum membrane of a cell, that is infected with the virus.

11. The method of claim 8, wherein the virus belongs to the Flaviviridae family.

12. The method of claim 11, wherein the infection is a Hepatitis C infection.

13. The method of claim 12, wherein said administering reduces production of lipid droplets in a cell that is infected with the Hepatitis C virus.

14. The method of claim 12, wherein said administering inhibits in interaction of the lipid droplets with a core protein of the Hepatitis C virus.

15. The method of claim 12, wherein said administering reduces an infectivity of the Hepatitis C virus.

16. The method of claim 8, wherein the virus belongs to the Retroviridae family.

17. The method of claim 16, wherein the virus is an HIV-1 virus.

18. The method of claim 1, wherein the composition further comprises at least one active agent encapsulated into the lipid particle.

19. The method of claim 18, wherein said administering results in delivering of the at least one active agent into an endoplasmic reticulum of a cell, that is infected with a virus causing the infection.

20. The method of claim 18, wherein the at least one active agent comprises an iminosugar.

21. The method of claim 18, wherein the at least one active agent comprises an alpha glucosidase inhibitor.

22. The method of claim 18, wherein the at least one active agent comprises an ion channel inhibitor.

23. The method of claim 18, wherein the at least one active agent comprises N-substituted deoxynojirimycin.

24. The method of claim 18, wherein the at least one active agent comprises N-butyl deoxynojirimycin.

25. The method of claim 18, wherein the at least one active agent comprises at least one anti-HIV agent.

26. The method of claim 18, wherein the at least one active agent comprises at least one anti-Hepatitis agent.

27. The method of claim 18, wherein the at least one active agent comprises at least one of an immunostimulating or immunomodulating agent and a nucleotide or nucleoside antiviral agent.

28. The method of claim 1, wherein the composition further comprises an antiviral protein.

29. The method of claim 28, wherein the antiviral protein is intercalated into a lipid layer or bilayer of the lipid particle or is conjugated with the lipid particle.

30. The method of claim 1, wherein the composition comprises a targeting moiety conjugated with the lipid particle or intercalated into a lipid layer or bilayer of the lipid particle.

31. The method of claim 30, wherein the targeting moiety comprises a gp120/gp41 targeting moiety.

32. The method of claim 30, wherein the targeting moiety comprises a sCD4 molecule.

33. The method of claim 30, wherein the targeting moiety comprises a monoclonal antibody.

34. The method of claim 30, wherein the targeting moiety comprises E1 or E2 targeting moiety.

35. The method of claim 1, wherein the host is a human.

36. A method of treating or preventing an HIV infection comprising
administering to a host in need thereof a composition comprising a lipid particle comprising at least one of PS lipids or PI lipids, wherein said lipid particle does not contain CHEMS lipids.

37. The method of claim 36, wherein the lipid particle is a liposome.

38. The method of claim 36, wherein the lipid particle further comprises PE lipids.

39. The method of claim 38, wherein the PE lipids comprise at least one of DOPE lipids or PEG-PE lipids.

40. The method of claim 36, wherein the lipid particle further comprises PC lipids.

41. The method of claim 36, wherein the composition comprises at least one anti-HIV agent encapsulated in the lipid particle.

42. The method of claim 41, wherein the at least one anti-HIV agent comprises an iminosugar.

43. The method of claim 41, wherein the at least one anti-HIV agent comprises an alpha glucosidase inhibitor.

44. The method of claim 41, wherein the at least one anti-HIV agent comprises N-substituted deoxynojirimycin.

45. The method of claim 44, wherein the at least one anti-HIV agent comprises N-butyl deoxynojirimycin.

46. The method of claim 36, wherein the composition further comprises a targeting moiety conjugated with the lipid particle or intercalated into a lipid layer or bilayer of the lipid particle.

47. The method of claim 46, wherein the targeting moiety comprises a gp120/gp41 targeting moiety.

48. The method of claim 46, wherein the targeting moiety comprises a sCD4 molecule.

49. The method of claim 46, wherein the targeting moiety comprises a monoclonal antibody.

50. A method of drug delivery comprising administering to a subject in need thereof a composition comprising a lipid particle comprising at least one polyunsaturated lipid.

51. The method of claim 50, wherein the lipid particle is a liposome.

52. The method of claim 50, wherein the lipid particle comprises at least one of a polyunsaturated PE lipid or a polyunsaturated PC lipid.

53. The method of claim 52, wherein the lipid particle comprises a polyunsaturated PE lipid and a polyunsaturated PC lipid.

54. The method of claim 52, wherein the lipid particle comprises polyunsaturated 22:6 PE lipid.

55. The method of claim 52, wherein the lipid particle comprises polyunsaturated 22:6 PC lipid.

56. The method of claim 52, wherein the lipid particle comprises polyunsaturated 22:6 PC lipid and polyunsaturated 22:6 PE lipid.

57. The method of claim 52 applied for treating or preventing a disease or condition caused by or associated with a virus.

58. The method of claim 57, wherein the virus belongs to the Flaviviridae family.

59. The method of claim 57, wherein the disease or condition is a Hepatitis C infection.

60. The method of claim 59, wherein said administering reduces HCV RNA replication.

61. The method of claim 57, wherein the virus is an ER-budding virus.

62. The method of claim 57, wherein the virus is a glycoprotein containing virus.

63. The method of claim 50, wherein the composition further comprises at least one active agent encapsulated into the lipid particle.

64. The method of claim 63, wherein the at least one active agent comprises an iminosugar.

65. The method of claim 63, wherein the at least one active agent comprises an alpha-glucosidase inhibitor.

66. The method of claim 63, wherein the at least one active agent comprises an ion channel inhibitor.

67. The method of claim 63, wherein the at least one active agent comprises N-substituted deoxynojirimycin.

68. The method of claim 63, wherein the at least one active agent comprises N-butyl deoxynojirimycin.

69. The method of claim 63, wherein the at least one active agent comprises at least one anti-Hepatitis agent.

70. The method of claim 63, wherein the composition further comprises an antiviral protein.

71. The method of claim 70, wherein the composition comprises a targeting moiety conjugated with the lipid particle or intercalated into a lipid layer or bilayer of the lipid particle.

72. The method of claim 50, wherein the subject is a human.

73. A composition comprising a lipid particle that comprises PS lipids.

74. The composition of claim 73, wherein the lipid particle is a liposome.

75. The composition of claim 73, wherein the lipid particle further comprises at least one of PE, CHEMS, PI, PC or SP lipids.

76. The composition of claim 75, wherein the lipid particle comprises PE lipids and a molar ratio between the PE lipids and the PS lipids ranges from 0.5:1 to 20:1.

77. The composition of claim 76, wherein the molar ratio between the PE lipids and the PS lipids in the lipid particle ranges from 1:1 to 10:1.

78. The composition of claim 75, wherein the PE lipids comprise DOPE lipids and PEG-PE lipids.

79. The composition of claim 75, wherein the lipid particle comprises PE, PI and PC lipids.

80. The composition of claim 73, wherein a molar concentration of the PS lipids in the lipid particle is at least 10%.

81. The composition of claim 80, wherein the molar concentration of the PS in the lipid particle is at least 20%.

82. The composition of claim 73, wherein the lipid particle further comprises PI lipids and wherein a combined molar concentration of the PS lipids and PI lipids in the lipid particle is at least 10%.

83. The composition of claim 82, wherein the combined molar concentration of the PS lipids and PI lipids in the lipid particle is at least 20%.

84. A composition comprising a lipid particle that comprises at least one polyunsaturated lipid.

85. The composition of claim 84, wherein the lipid particle is a liposome.

86. The composition of claim 84, wherein the lipid particle comprises at least one of polyunsaturated PE lipid or polyunsaturated PC lipid.

87. The composition of claim 84, wherein the lipid particle comprises polyunsaturated PE lipid and polyunsaturated PC lipid.

88. The composition of claim 84, wherein the lipid particle comprises polyunsaturated 22:6 PE lipid.

89. The composition of claim 84, wherein the lipid particle comprises polyunsaturated 22:6 PC lipid.

90. The composition of claim 84, wherein the lipid particle comprises polyunsaturated 22:6 PC lipid and polyunsaturated 22:6 PE lipid.

91. The composition of claim 84, wherein a molar concentration of the polyunsaturated lipids in the lipid particle is at least 20%.

92. A method comprising contacting a cell with a lipid particle comprising a) at least one of PI or PS lipids and b) at least one labeled lipid comprising at least one label.

93. The method of claim 92, wherein the lipid particle is a liposome.

94. The method of claim 92, wherein the lipid particle further comprises at least one of PE and CHEMS lipids.

95. The method of claim 94, wherein the lipid particle comprises PE lipids and a molar ratio between the PE lipids and the PS lipids ranges from 0.5:1 to 20:1.

96. The method of claim 95, wherein the molar ratio between the PE lipids and the PS lipids in the lipid particle ranges from 1:1 to 10:1.

97. The method of claim 94, wherein the PE lipids comprise DOPE lipids and PEG-PE lipids.

98. The method of claim 92, wherein the lipid particle comprises PS, PE, PI and PC lipids.

99. The method of claim 92, wherein the virus is an ER-budding virus.

100. The method of claim 99, wherein the virus is an HCV virus or HBV virus.

101. The method of claim 92, wherein the at least one labeled lipid comprises a fluorophore-lipid conjugate.

102. The method of claim 92, wherein the at least one labeled lipid comprises a biotin-lipid conjugate.

103. The method of claim 92, wherein said contacting results in labeling the ER membrane of the cell with the label.

104. The method of claim 92, wherein the cell is a cell infected with an ER budding virus and wherein said contacting results in labeling a viral particle that buds that the cell with the label.

105. The method of claim 104, further comprising purifying the labeled viral particle.

106. The method of claim 104, further comprising imaging the labeled viral particle.
